SEQUENCE LISTING (9 sequences)

SEQ ID NO: 1 - DNA, 681 bp, Bovine viral diarrhea virus
  CDS (1)..(681): BVDV Erns
gagaacataacgcaatggaacttacaagataatggaacggaaggcatacaacgagccatgtttcaaagaggagtgaatagaagtttacatgggatctggccagagaaaatctgtacaggtgttccttcccacctggccactgacacagaattgaaggcaattcatggtatgatggatgcaagtgagaagacaaattatacgtgctgcagactccaacgccatgaatggaacaaacatggttggtgcaactggtacaacattgaaccttggatcctccttatgaataaaactcaggccaaccttactgagggtcagccactaagggagtgtgccgtcacatgccggtatgatcgagatagtgacctgaatgtagtaacacaagccagggatagccccacaccattaacaggttgcaagaaaggcaaaaacttttcctttgcaggcatattggtacaagggccttgcaactttgaaatagccgtaagtgatgtgctgttcaaagagcatgattgcactagtgtgattcaagacacagctcactacctcgtagacgggatgaccaactccctagagagtgccaggcaagggaccgcgaaactaacaacttggctgggcaggcagcttgggatactaggaaagaaactggaaaacaagagtaagacatggtttggggcatatgca

SEQ ID NO: 2 - protein, 227 aa, Bovine viral diarrhea virus
ENITQWNLQDNGTEGIQRAMFQRGVNRSLHGIWPEKICTGVPSHLATDTELKAIHGMMDASEKTNYTCCRLQRHEWNKHGWCNWYNIEPWILLMNKTQANLTEGQPLRECAVTCRYDRDSDLNVVTQARDSPTPLTGCKKGKNFSFAGILVQGPCNFEIAVSDVLFKEHDCTSVIQDTAHYLVDGMTNSLESARQGTAKLTTWLGRQLGILGKKLENKSKTWFGAYA

SEQ ID NO: 3 - DNA, 666 bp, Bungowannah virus
  CDS (1)..(666)
accaacgtgacacaatggaacttatgggacaataaaagtactacagacatacatagcgtcatgttttctagagggattaaaaggagtctgcatggaatttggcccacacaaatctgcaaagggatccctacacatctagcagcagactatgaactgaaaaggattcacgggatggtggatgcaagccccatgaccaacttcacatgttgtaggctacagagacatgagtggaacaagcatgggtggtgcaactggtacaatatagagccgtggatcaatctcatgaataatacccaaggactattaaacactggagacaatttcactgagtgcgcagtcacatgcaggtatgatgcagacttaggggtgaatatagtgactcaagccaggactactccaactatcctgactggctgtaagaaagggcacaacttctctttctcaggggaggtcagggcctcaccctgcaactttgagttaactgctgaagacttgctcaggatcatggatcacaccaactgcgagggatttacctacttcggggaaggaatcgttgacggttacaccgaggtagtagagaaggccaggtcaagtggtttcagggctctcacatggttgtcgagtaagattgaaaacaccaagaaaaaaatattcggagctgaagcc

SEQ ID NO: 4 - protein, 222 aa, Bungowannah virus
TNVTQWNLWDNKSTTDIHSVMFSRGIKRSLHGIWPTQICKGIPTHLAADYELKRIHGMVDASPMTNFTCCRLQRHEWNKHGWCNWYNIEPWINLMNNTQGLLNTGDNFTECAVTCRYDADLGVNIVTQARTTPTILTGCKKGHNFSFSGEVRASPCNFELTAEDLLRIMDHTNCEGFTYFGEGIVDGYTEVVEKARSSGFRALTWLSSKIENTKKKIFGAEA

SEQ ID NO: 5 - DNA, 687 bp, Artificial Sequence
  Heterologous Pestivrus Erns gene Bungowannah + BVDV1 CP7
  CDS (1)..(687)
accaacgtgacacaatggaacttatgggacaataaaagtactacagacatacatagcgtcatgttttctagagggattaaaaggagtctgcatggaatttggcccacacaaatctgcaaagggatccctacacatctagcagcagactatgaactgaaaaggattcacgggatggtggatgcaagccccatgaccaacttcacatgttgtaggctacagagacatgagtggaacaagcatgggtggtgcaactggtacaatatagagccgtggatcaatctcatgaataatacccaaggactattaaacactggagacaatttcactgagtgcgcagtcacatgcaggtatgatgcagacttaggggtgaatatagtgactcaagccaggactactccaactatcctgactggctgtaagaaagggcacaacttctctttctcaggggaggtcagggcctcaccctgcaactttgagttaactgctgaagacttgctcaggatcatggatcacaccaactgcgagggatttacctacttcggggaaggaatcgttgacggttacaccgaggtagtagagagtgccaggcaagggaccgcgaaactaacaacttggctgggcaggcagcttgggatactaggaaagaaactggaaaacaagagtaagacatggtttggggcatatgca

SEQ ID NO: 6 - protein, 229 aa, Artificial Sequence
  Synthetic Construct
TNVTQWNLWDNKSTTDIHSVMFSRGIKRSLHGIWPTQICKGIPTHLAADYELKRIHGMVDASPMTNFTCCRLQRHEWNKHGWCNWYNIEPWINLMNNTQGLLNTGDNFTECAVTCRYDADLGVNIVTQARTTPTILTGCKKGHNFSFSGEVRASPCNFELTAEDLLRIMDHTNCEGFTYFGEGIVDGYTEVVESARQGTAKLTTWLGRQLGILGKKLENKSKTWFGAYA

SEQ ID NO: 7 - DNA, 40 bp, Artificial Sequence
  PCR primer
ctttcaagtcacaatgggaaccaacgtgacacaatggaac

SEQ ID NO: 8 - DNA, 48 bp, Artificial Sequence
  PCR primer
cgcggtcccttgcctggcactctctactacctcggtgtaaccgtcaac

SEQ ID NO: 9 - DNA, 13743 bp, Artificial Sequence
  cDNA of mutant Pestivirus pBVDV-1_dNpro_Erns-Bungo/CP7
gctagctaatacgactcactatagtatacgagaattagaaaaggcactcgtatacatattggacactctaaaaaaaaaaaataattaggcctaggggacaaaaatcctccttagcgaaggccgaaaagaggctaaccatgcccttagtaggactagcaaaataaggggggtagcaacagtggcgagttcgttggatggctgaagccctgagtacagggtagtcgtcagtggttcgacgctttggaggacaagcctcgagatgccacgtggacgagggcatgcccacagcacatcttaacctggacaggggtcgttcaggtgaaaacggtttaaccaaccgctacgaatacagtctgataggatgctgcagaggcccactgtattgctactgaaaatctctgctgtacatggcacatggagttgatcacaaatgaacttttatacaaaacattaattaactccgacacaaaagatgaaggggtggtgaggaagaagcaacaaaagccagataggttggaaaaggggagaatgaagataacacctaaggagtcagagaaagacagtaagaccaagccgccagatgctacgatagtggtagatggagtcaagtatcaggtaaagaaaaaaggaaaagtcaagagcaagaacacccaggacggcttataccacaacaaaaataaacctcaagagtcgcgcaagaaactagagaaagccctattggcctgggcaataatagccctggttttctttcaagtcacaatgggaaccaacgtgacacaatggaacttatgggacaataaaagtactacagacatacatagcgtcatgttttcgagggggattaaaaggagtctgcatggaatttggcccacacaaatctgcaaagggatccctacacatctagcagcagactatgaactgaaaaggattcacgggatggtggatgcaagccccatgaccaacttcacatgttgtaggctacagagacatgagtggaacaagcatgggtggtgcaactggtacaatatagagccgtggatcaatctcatgaataatacccaaggactattaaacactggagacaatttcactgagtgcgcagtcacatgcaggtatgatgcagacttaggggtgaatatagtgactcaagccaggactactccaactatcctgactggctgtaagaaagggcacaacttctctttctcaggggaggtcagggcctcaccctgcaactttgagttaactgctgaagacttgctcaggatcatggatcacaccaactgcgagggatttacctacttcggggaaggaatcgttgacggttacaccgaggtagtagagagtgccaggcaagggaccgcgaaactaacaacttggctgggcaggcagcttgggatactaggaaagaaactggaaaacaagagtaagacatggtttggggcatatgcagcctctccctattgtgaggtagaacggaagcttggttacatctggtatacaaagaattgcactccagcctgtttgcctaggaatacaaagatcatcggccccggtaggtttgacaccaatgccgaggatggtaaaatactgcatgagatggggggtcacttgtcggaggtgctactactctcagtggtagtgctgtccgatttcgctccagagacagccagtgtgatatacttgattcttcatttctccatcccacaaggacacactgatatacaagattgtgacaaaaaccaactaaacctcaccgtagaactcacaacagcagaagtaataccaggctcagtttggaacttgggtaaatatgtttgtgtaagaccagattggtggccttatgagacagccacagtcctggtgattgaagaggtgggtcaagtaattaaggttgtcttaagggcgttaaaagatctgacgcgcatttggaccgctgctacaaccactgcattcttggtttgtctggtgaaggtagtgagaggccaagtgttacaaggtatactgtggctgatgctcataacaggggcgcaaggatatccagactgcaaacccggcttttcatacgccatagccaaaaatgatgagattggcccacttggagctacaggcctcaccactcagtggtacgaatactcggatgggatgcggctgcaggactcagtagttgaagtttggtgtaaaaatggagagatcaaatatctaatcagatgcgggagggaagccaggtatctggctgttctacacacgagagccttgccgacatctgtagtatttgaaaaaatttttgatgggaaagaacaagaggacatagtagaaatggatgacaactttgaattcggtctttgcccgtgtgatgctagacccttgataaggggaaaatttaatacaacacttctaaatgggccagccttccagatggtttgccctataggatggacagggactgtaagctgtacactggccaataaggatacgttagccacaatcgttgtgagaacgtataagagggtcaggccttttccatataggcaggactgtgtcacccagaaaaccatcggggaagacctctacgactgtgccttaggagggaattggacttgtgtgccgggggatgcactacgatatgtagctgggcccgttgagtcttgtgagtggtgtggttacaagtttttaaaaagtgagggtctgccgcatttcccaatcggcaaatgcaggctgaagaatgagagtggctatagacaagtggatgagacttcttgcaacagaaacggcgtggctatagtgccatctggcacggtcaaatgcaagataggggacacggtggtgcaagtcattgcaatggatgataaactagggcctatgccttgcaaaccacatgaaatcatatccagtgaggggccagtggaaaagacggcatgcaccttcaactacacaagaacattaaaaaacaagtactttgagcccagggataactattttcaacaatacatgttaaagggggagtaccaatattggtttgacctagagatcactgaccaccaccgagattacttcgctgagtccctgttggtgatagtagttgcactcctgggtggcaggtacgtgctttggctgctggtcacatacatgatcttatcagaacagatggcctcgggtgtccagtatggggcaggtgaaatagtgatgatgggcaacttgttaacacatgacagtgttgaagtggtgacatatttcttactactatacctactactaagagaggaaaacaccaaaaaatgggtcatacttatataccacatcatagtaatgcatcctctaaaatcggtgacggtgatattgctaatggttggggggatggcaaaggctgaaccaggtgcccaggggtacctagagcaggtagaccttagttttacgatgattacgatcatcgtaataggtctggttatagctaggcgtgatcccactgtggtgccactagtcactatagtcgcggcactgaagatcacaggactaggctttgggcccggagtggatgcagctatggcagttctcaccttaaccctactgatgactagttatgtgacagactacttcaggtataaaaggtggatacaatgtatcctcagcttagtagccggggtgttccttatccggaccctcaaacatctaggtgaactcaaaacccctgagctgaccataccaaattggaggccactaaccttcatactattatacctgacttcagcaacagttgttacaagatggaaaattgatatagctggcatattcctgcaaggggcccctatccttttgatgatcgccaccctatgggctgacttcttgactcttgttctgatcctacccacctacgaattagccaagctgtactacctaaagaacgtcaagactgacgtggagaagagttggggtgttccttatccggaccctcaaacgctgggggggttagactacaggacaattgactctgtctatgatgtggatgaaagtggagaaggcgtgtacctcttcccgtccagacagaagaaaaataagaatatcagcatactcttgcccctcatcagagctacgctaataagttgtattagcagcaaatggcagatggtgtatatggcttacttaaccctggactttatgtactacatgcacagaaaggttattgaagagatatcagggagtaccaatgtgatgtctagagtgatagcagcacttatagaattaaactggtccatggaagaagaagagagcaagggcttaaagaagttttttatactatctggaaggttgaggaaccttataataaagcataaggttaggaaccagactgtggcaagctggtatggggaggaagaagtctacggcatgccaaaagtcgtaaccataataagggcctgcacgctaaacaagaacaaacattgcataatatgcacagtatgtgaggctagaaagtggaagggaggcaactgccctaaatgcggccgccacgggaagcccatcatttgtgggatgactctagcggattttgaagaaaggcactacaagagaatttttataagggaaggtaactttgaaggacccttcaggcaggaatacaatgggtttgtacaatacaccgctagggggcaattgttcctgagaaatttacccatattggcaaccaaagtaaaaatgatcatggtaggcaacctaggagaggaaatcggtgatctagaacacctaggatggatcctaaggggacctgccgtgtgcaagaaaataactgagcacgaaaaatgccatgtcaacatactggacaagctgactgcgttttttggagttatgccaagagggactacaccaagggctccggtgagattcccaacagcactactaaaggtaaggaggggattggaaaccggttgggcttacacgcatcaaggtggcataagctcagtagaccatgtgaccgctggcaaggatctattggtttgtgacagtatgggtagaactagagtggtttgccaaagcaacaacaagttaactgatgagacagaatatggtgtcaagacggactccggatgtccagatggtgccagatgctatgtattaaacccagaggcagtaaatatatcagggtccaagggagctgtcgtacacctccaaaaaacgggtggggaatttacatgtgttactgcatcaggtacaccggccttcttcgacctgaaaaatttgaaaggatggtcgggtctacccatatttgaagcctccagcggcagagtggttggcagagtcaaagtgggaaagaatgaggaatccaaacccacaaaattaatgagtggtatccaaactgtttctaaaaatacggccgatttaacagaaatggtcaagaagataaccagcatgaacaggggggactttaggcagataacccttgcaacaggggcagggaagaccactgagctcccaaaagcagtgatagaggagataggacgacacaaacgggtactagtgctcataccattaagagcagcagctgagtcagtctatcaatacatgagattgaaacacccaagtatctcctttaacctgagaataggggacatgaaagaaggggatatggcaaccgggatcacctacgcctcatatggatatttttgccaaatgccacaaccaaagctcagagcagcaatgatagagtattcatacatatttctggatgagtatcactgcgctactcctgagcagttggctgttataggaaaaattcacagattttctgagagcataagagtggttgccatgactgccaccccagcagggtcagtaaccacaacagggcaaaaacacccaatagaagaattcatagcccctgaggtgatgaaaggggaggaccttggaagccagttccttgacatagcggggttaaagatccctgtagaggagatgaagggtaacatgttggttttcgtgcccacgaggaacatggcagttgaagtagccaagaaactaaaagccaagggctacaactcagggtattactacagtggggaagacccagctaacttgagagtggtaacatcacagtccccatacgtcgtggtagccactaatgccatcgagtcaggggtaacgctgccagatttagatacagttgttgacacaggtctgaaatgtgagaagagggtgagggtgtcttccaaaataccctttatagtaacaggccttaagagaatggctgtcactgtgggcgaacaggctcagcggagaggcagggtaggtagagtgaaacccggtaggtattatagaagtcaggaaacagcaaccgggtcaaaggactaccactatgacttgttacaggcacagaggtacgggatcgaagatgggatcaacgtaacaaagtcctttagggagatgaattatgactggagcctgtatgaggaagacagcttgctgataacccagctggagatactgaacaatctactcatctctgaagatttaccagcagctgttaaaaacatcatggcaagaactgatcacccagagcctatccagcttgcatataacagttatgaggtccaagtccctgtgctgttcccaaaaataaggaatggggaggtcacagacacttacgagaactactcattcctaaatgcaaggaaactaggggaagacgtgcccgtgtacgtttatgccaccgaagatgaagatctggctgtggaccttctaggcttggactggccagacccagggaatcagcaagtagtggagactgggaaggcactgaagcaagtggtaggactgtcctctgccgaaaatgccttgctcatagccctatttgggtatgtaggataccaagccttgtcaaaaagacacgtcccaatgatcacagacatatacactatagaagatcaaagactagaggacacaacccaccttcaatatgcgcccaatgccataagaactgaggggaaggagactgaactaaaggaattagcagtgggtgacttggacaaaatcatgggttccatctcggactatgcatcagagggattgaatttcgtaaggtcccaagcagaaaagatgagatctgcccccgctttcaaagaaaacgtggaagctgctaaagggtacgtccaaaagtttattgattctctcatagaaaataaagaaaccataatcagatatggcctgtggggaacacacacggcactctacaagagtattgccgcgagattgggtcatgaaactgcattcgctacactagtgataaagtggctggccttcgggggtgagtcggtgtcagaccacatgagacaagcagctgtcgacctggttgtttattatgtgatcaataagccctccttcccaggggattctgaaacccaacaggaaggaaggcgattcgtcgccagcctgttcatctccgctttggcaacctacacatacaaaacttggaattacaacaacctctccaaggtagtagaaccagccttagcatacctcccctatgctaccaatgcactaaaaatgtttaccccgaccagactggagagcgtagttatacttagtaccacaatatacaaaacttacctctcaataaggaagggaaagagtgatggactgttgggtacagggatcagtgcagcaatggagattctatcacagaacccagtgtcggtaggtatatctgtcatgctgggggtgggggcgattgccgcgcacaatgccattgagtctagtgaacaaaaaaggaccctgttgatgaaagtgtttgtaaaaaacttcctggaccaggcggcaacagatgagctggtaaaggaaaacccagagaaaataataatggccctatttgaagcagtccagacaattggcaaccccttgaggctcatatatcacctgtatggggtttactacaaaggctgggaagcaaaagaactatcagagagaacagcaggcaggaacctgttcaccttgataatgttcgaagccttcgagctactagggatggactctgaagggaagataaggaacctgtctgggaattatgtcctggatttgatctacagcctacataaacagataaatagaggcttgaaaaaaatagtcttggggtgggctcccgcaccatttagttgcgactggactcctagtgatgagagaattaggttacccacaaacaactatctaagagtagaaactaagtgtccatgtggctatgagatgaaagcactaaggaacgttggtggcagtcttaccaaagtggaggagaaaggaccttttctctgcaggaacaggcttggtagagggccggtcaactatagagtcacaaagtactatgatgacaacctcaaagagataaaaccagttgctaaactagaaggatttgtggatcactattacaaaggtgttacagcaaggatagattatggcagagggaaaatgctattagctactgataaatgggaggtggagcacggtgttgtcactaggttggcaaagagatataccggagttggattcaagggagcatacctgggtgatgaacccaaccaccgcgacctagtagaaagagactgtgcaactataacaaaaaatacagtgcagtttttaaaaatgaagaaaggctgtgcatttacctatgacttaaccctgtccaatttaaccaggttaattgaattggtacacaaaaataacctagaagagaaagacataccagcagccacagtaacgacatggctggcttatacttttgtaaatgaagatattgggactataaaaccagtactaggagagagagtggtcaccgacccagtggtggatgttaacttacaaccagaagtacaagtggatacatcagaggttgggatcactttagttggtagggcagccttaatgacgacaggtactacacccgtagtcgaaaaaacagagcccaatgctgatggtggtccaagctccataaagattgggttggatgaaggaagatacccaggacctggactgcaagaccgcaccttgaccgatgaaatacattctagggatgaaaggccctttgttctagtcctgggctcaaaaaattctatgtcaaatagagctaaaactgctagaaacatcaacttatacaaggggaataaccccagggagattagagatctgatggcacaggggcgtatgctagttgtggccttaaaggattttaaccctgagttgtctgaactagttgatttcaaggggactttcttagacagggaagccttggaagctctcagcctggggcggccaaagtccaagcaggtgaccacagccacagttagggagttattagagcaggaggtacaagttgagatccccagttggtttggagcaggtgatccagtcttcttggaagtgactttgaagggtgacagatatcacttagtaggagatgtagatagagtgaaagatcaagcgaaggagcttggggccacggaccagacaagaatagtgaaggaagtgggtgcaagaacctataccatgaagctgtctagttggtttcttcaggcaacaaataaacagatgagcttgacccctttatttgaggagctattgctacgttgcccccctaaaataaagagcaataaagggcacatggcatcagcttaccaactagcacagggaaactgggagccccttgactgtggagttcacctgggcaccatacctgccaggagggtaaaaatccacccatatgaagcttacctgaaactgaaggatttattggaagaagaagaaaagaaaccaaagtgtagagacacagtaataagagaacacaacaagtggatcctcaaaaaagtgaggcaccagggtaatctcaatacaaagaaaatcctcaaccctggaaagctatcagaacagctagatagagaagggcataaaagaaacatttataacaatcagattggcaccataatgacggaagcaggaagtaggttggaaaaattaccagtcgtcagagcccaaactgacactaaaagcttccatgaggcaatcagagataagatagacaagaatgaaaatcagcagagcccaggactgcatgataaattgttagagatctttcatacaatagcccaacccagcctaagacacacctacagtgacgtgacgtgggagcaacttgaggcaggggttaatagaaagggggctgctggctttctagagaagaagaatgttggagaagtactggactcagagaagcacctggtggaacaactgatcagagatttgaaaacaggaaggaagataagatattatgagacagcaataccaaaaaatgagaagagagatgtcagtgatgattggcaatcaggggacttagtagatgagaagaaaccaagggtgattcaataccctgaagctaaaacaagactagccatcactaaagtaatgtacaactgggtgaaacagcagcccgtcgtgatcccagggtatgaagggaagaccccattatttaacattttcaacaaggtgaggaaggaatgggatttgttcaatgaaccagtagctgtgagtttcgacactaaggcttgggacacccaagtaactagtagagatctacggcttattggtgaaattcaaaaatattactacaggaaagagtggcacaaattcatcgataccattactgaccatatggtggaggtgcccgtcataacggcagatggtgaggtatacataagaaatggacaaaggggtagtggccagccagacacaagtgcaggcaatagcatgctaaacgtgttaacaatgatgtatgccttctgtgaaagtacgggggttccatacaagagtttcaatagggttgcaaggatccatgtctgtggggatgacggcttcctaataacagagaaggggctgggattaaagtttgccaacaatgggatgcaaattctgcacgaagcaggcaagcctcaaaagataactgagggggaaagaatgaaagttgcctataggttcgaggacatagaattctgctctcatacaccagtccccgttaggtggtctgataacaccagcagttacatggccggcagagacactgccgttatattatcaaagatggcaacaagattggattcaagtggagaaaggggtactatagcatatgaaaaagcagtggcctttagttttttgctgatgtactcctggaatcctcttgtgaggaggatctgtctactggtcctttcacagcagccagagacaactccatcaacccagaccacttactattataaaggagacccaataggagcctacaaagatgtaataggtaagaatttgtgtgaattaaaaaggacgggttttgaaaaattggccaatttaaacctaagcctgtccacgttaggaatctggtccaaacatacaagtaaaagaatcatccaagactgtgtaaccatcgggaaagaggaaggcaattggctggtcaatgccgacaggttgatatctagcaaaactggccatttgtacatacctgacaaaggttatacattacaagggaaacattatgaacaacttcaactgcaggcaagaactagcccagtcacgggagtagggacggagagatataaactaggccctatagtaaacctgctgctgaggaggttgagagttctgcttatggcagctgtcggtgccagcagttgaaataatgtatgtatatattgtatataaatctgtatttgtatatattatgtttaaatttagttgagattagtagtgatatatagttatctacctcaagctaacactacactcaatgcacacagcactttagctgtatgagggtacacccgacgtcacccccgggggatccactagttctagagcggccattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagaactagtggatcccgcgcg

(19) United States
(12) Patent Application Publication
(10) Pub. No.: US 2019/0240311 A1
(43) Pub. Date: Aug. 8, 2019

(54) PESTIVIRUS MARKER VACCINE

(71) Applicant: Intervet Inc., Madison (JP)

(72) Inventors: Ilona REIMANN, Gristow (DE); Patricia KONIG, Sundhagen OT Kirchforf (DE); Martin BEER, Neuenkirchen (DE)

(73) Assignee: Intervet Inc., Madison, NJ (US)

(21) Appl. No.: 16/066,218

(22) PCT Filed: Dec. 23, 2016
(86) PCT No.: PCT/EP2016/082537

§ 371 Date: Jun. 26, 2018

(30) Foreign Application Priority Data
Dec. 30, 2015 (EP) 15203202.5

(51) Int. Cl. A61K 39/12

(52) U.S. Cl. CPC A61K 39/12; C12N 2770/24321; C12N 2770/24322; A61K 2039/5254; C12N 2770/24371; C12N 2770/24362; C12N 2770/24334

(57) ABSTRACT

The present invention provides a mutant Pestivirus comprising a chimeric Erns gene, which provides the mutant Pestivirus with the capacity to evade serologic detection, but maintains good vaccine properties, and viral replication.

The present invention relates to the fields of veterinary virology and -vaccinology. More specifically the invention relates to a mutant Pestivirus with a mutated Erns gene, to vaccines and medical uses of that mutant Pestivirus, to methods of preparation of the mutant Pestivirus and the vaccines, and to diagnostic methods using the mutant Pestivirus or its mutated Erns gene.

The Pestivirus genus of the Flaviviridea family contains a number of animal pathogenic viruses that are of considerable economic relevance to the agricultural industry. Pestiviruses occur worldwide, and can infect different species of animals within the Artiodactyla. Main virus members are bovine viral diarrhoea virus type (BVDV), infecting ruminants and swine, classical swine fever virus (CSFV) infecting swine, and border disease virus, infecting ruminants and swine. There is a variable extent of serological cross-reaction between different Pestiviruses, which causes much difficulty in their serodiagnosis.

The Pestiviral virion is enveloped and comprises a nucleocapsid with a single-stranded, linear, positive-sense RNA genome of about 12 kb. The genome encodes 4 structural and 8 non-structural proteins, and is translated into one large polyprotein of about 3900 amino acids, which is then cleaved by viral- and host proteases. An extensive review of Pestiviral characteristics is given in the chapter on Flaviviruses in Fields Virology (4th Edition 2001, Lippincott Williams & Wilkins, ISBN-10: 0781718325).Pestivirus molecular biology is reviewed in Tautz et al. (2015, Adv. in Virus Res., Vol. 93, Chapter 2, p. 47-160).

A review of the characteristics of Pestivirus glycoproteins is given in Wang et al. (2015, Viruses, vol. 7, p. 3506-3529). The immunodominant proteins are the envelope glycoproteins Erns and E2, and the non-structural protein NS3; of these, E2 induces virus-neutralising antibodies. The Erns envelope glycoprotein is unique to viruses of the genus Pestivirus, and has a number of functions: at its N- and C-termini there are cleaving signals for releasing it from the polyprotein. The centre region of Erns protein is associated with an RNAse activity, which can interfere with double stranded RNA, and in this way influences the Interferon response by the infected host cell. The C-terminal side of the Erns protein has a membrane association signal. Currently known Erns genes are between 666 and 681 nucleotides in length, encoding an Erns protein of between 222 and 227 amino acids. In the literature the Erns protein is also called E0 (E zero), gp48 or gp44-48.

Within the Pestivirus genus there is a core group of viruses that are closely related serologically and genetically. This core group consists of the four official viral species: BVDV-1, BVDV-2, CSFV, and border disease virus. With regard to relatedness on the basis of the Erns protein, some non-official species also fall within that group: isolates from Reindeer and Giraffe, and the HoBi Pestiviruses (also named HoBi-like, or BVDV-3). See: Hause et al. (2015, J. of Gen. Virol., vol. 96, p. 2994-2998), who present an overview of the relatedness of the currently known Pestivirus ‘species’; the relatedness of Erns proteins is presented in FIG. 1, panel C on page 2997 of that reference.

Several isolates or (partial) genomes are known of Pestiviruses that are more distantly related, serologically and/or genetically, from this core group and more continue to be discovered. In order of increasing distance in relationship to the core group on the basis of the Erns protein, these are: Antelope Pestiviruses (also named: Pronghorn); Bungowannah virus; Norway rat Pestivirus (NRPV); and most distant are atypical porcine Pestivirus (APPV), and Rhinolophus affinis Pestivirus (RaPV).

The Bungowannah Pestivirus was identified in 2003 in Australia, as the cause of myocarditis, stillbirths and mortalities in swine. A characterisation of Bungowannah virus is given in Kirkland et al. (2015, Vet. Microbial., vol. 178, p. 252-259). Also the Bungowannah virus is a subject of WO 2007/121.522.

The Norway rat Pestivirus is described by Firth et al. (2014, Mbio, vol. 5, e01933-14); APPV is described in Hause et al., 2015 (supra); and RaPV is described by Wu et al. (2012, J. of Virology, vol. 86, p. 10999-11012).

CSFV causes classical swine fever or hog cholera, a severe haemorrhagic disease that is often fatal for porcine animals. This severe clinical disease causes much animal suffering, and considerable economic losses to sectors dependent on commercial pig farming and their products. Vertical transmission of CSFV is possible by transplacental infection of a foetus. In addition, pigs can become chronically infected, causing persistent horizontal spread of the virus.

Bovine viral diarrhoea virus (BVDV) is the causative agent of one of the most widespread viral diseases of cattle. The virus is endemic in most cattle populations worldwide, and causes a variety of symptoms, of which the reproductive and respiratory diseases are most prominent.

BVDV is biologically diverse, in having different genotypes and biotypes. The genotypes: BVDV-1 and -2, are now considered as separate species, and have genetic differences in the structural glycoproteins E1 and E2. Within both species BVDV-1 and -2, strains of high- or of low virulence have been described. Several sub-genotypes have developed and are found in the field, their prevalence varies; currently relevant are 1b, 1f, 2a, and 2c.

The difference in BVDV biotype: either cytopathogenic (cp) or non-cytopathogenic (ncp), is determined by a genetic difference in the non-structural genes NS2 and NS3. While both biotypes may cross the placenta and infect the foetus, only the ncp form may cause persistently infected (PI) calves. The birth of PI calves is the cornerstone of BVDV epidemiology, as these animals will generally be asymptomatic for some time, but spread infectious BVDV virus life-long, contaminating their herds and their surroundings. Also these PI animals develop a fatal BVDV disease, often within a year, the so-called ‘mucosal disease’. The cp type BVDV is considered to evolve in PI animals, through a mutation of the NS2 gene. While the cp biotype causes more acute symptoms, it is easier to clear for a host animal than the ncp biotype.

For an overview of BVDV related diseases: abortion, stillbirth, haemorrhagic syndrome, and mucosal disease, see “The Merck veterinary manual” (10th ed., 2010, C. M. Kahn edt., ISBN: 091191093X).

Vaccination against Pestivirus infection and/or their induced disease is common practice and many types of vaccines are available commercially. Such vaccines can be based on live (i.e. replicating) or inactivated Pestivirus, or even on viral subunits.

In several countries there are governmental programs for the control of Pestiviruses, such as emergency vaccination and/or culling of infected animals. For BVDV the detection and elimination of PI animals, together with foetal protection by vaccination are important. However eradication is complicated by reinfection from wild animal reservoirs. Also, transport across borders may be restricted for animals that are seropositive for antibodies against a Pestivirus; this interferes with the application of general vaccination regimes.

Therefore, efforts have focussed on the development of vaccines that allow the serological “differentiation of infected from vaccinated animals” or: DIVA. The basic principle behind this type of discriminating test is the vaccination of a target animal with a vaccine that has a positive (an additional feature) or a negative (a missing feature) ‘marker’ function, which can be differentiated serologically from the infection of an animal with the wild type micro-organism. For example the marker vaccine may be deficient in one or more antigens that are present in the wild type micro-organism. An infected host will then become seropositive for that antigen, while vaccinates remain seronegative for that antigen in a suitable assay system.

In the use of diagnostic tests to monitor eradication- and control programs, it is critical to have a sufficient level of sensitivity and specificity of the test, as faults in this respect can have grave consequences either way: false positives can cause unnecessary quarantine or culling of animals, and false negatives may cause spread of a pathogen by transport of undetected carrier-animals across borders. In case of doubt, negative scoring animals may be re-tested after some weeks.

While differentiation between vaccine and field-virus is generally possible using molecular biological techniques, for instance using PCR, it is usually preferred to apply the DIVA principle via some sort of immuno-diagnostic assay. Such assays can be applied even at considerable time after infection, at large scale, and are relatively cheap.

Much used diagnostic tests are enzyme immunoassays, such as ELISA's. Commercial ELISA tests for Pestiviruses are commonly based on one of the immunodominant proteins: Erns, E2, or NS3, and will detect either the antigen or antibodies against it. Examples are:

For BVDV: the PrioCHECK® BVDV Antibody ELISA Kit (Thermo Fisher), an inhibition ELISA for BVDV NS3; and the IDEXX BVDV PI X2 Test, an ELISA for BVDV Erns antigen detection.

For CSFV: CSFV E2 Antibody Test Kit (Biocheck), an indirect ELISA for detecting antibodies to CSFV E2; CSFV Ab Test (IDEXX);); the PrioCHECK® CSFV Erns ELISA, detects CSFV Erns-specific antibodies; and PrioCHECK® CSFV Antigen ELISA Kit® (Thermo Fisher), a double antibody-sandwich direct Elisa for CSFV antigen.

Several live attenuated Pestivirus vaccines have been described so far. Although these vaccine viruses can be differentiated from field virus by genetic testing, however they have inadequate serologic marker capability. For instance for BVDV, live attenuated vaccine strains have been described in: WO 2005/111.201, describing a Pestivirus mutant (preferably a BVDV of the ncp biotype) having mutations in the Npro and in the Erns genes; and: WO 2008/034.857, describing a Pestivirus mutant of the cp biotype wherein part of the Npro gene has been deleted. See also: Zemke et al. (2010, Vet. Microbiol., vol. 142, p. 69-80).

WO 2014/033.149, describes a Pestivirus having a mutation in an epitope of helicase domain 2 from the NS3 protein.

Similarly, van Gennip et al. (2001, Vaccine, vol. 19, p. 447-459) described live chimeric CSFV carrying a BVDV Erns or E2 gene to evade recognition by anti-CSFV antibodies. However residual serologic cross-reactivity with anti-Erns anti-sera still caused false positive reactions.

Finally, a CSFV marker vaccine was licensed in Europe which consisted of a BVDV backbone expressing the CSFV E2-protein (Suvaxyn™ CSF marker, Zoetis).

As a result, there are very few options for live attenuated Pestivirus marker vaccines. One of the reasons is that problems were encountered in balancing the properties of the vaccine virus of having a good virus replication and providing effective immune protection, with having a clear and detectable serologic difference with wild type virus.

An example is Luo et al. (2012, Vaccine, vol. 30, p. 3843-3848, and WO 2010/064.164). These authors exchanged the complete BVDV Erns gene, by the Erns gene from Pestiviruses, such as from Reindeer, Giraffe, or Pronghorn Antelope. The authors report that serological cross-reactivity was most reduced upon use of an Erns gene from their most distant donor Pestivirus: Pronghorn Antelope. However the BVDV-Pronghorn Erns chimeric virus also replicated the worst of all candidates tested.

It is therefore an object of the present invention to overcome a disadvantage in the prior art, and to accommodate to a need in the field by providing improved Pestivirus marker vaccine viruses, that lack specific serologic cross-reactivity with other Pestiviruses, but still have good viral replication, and induce effective immune-protection against Pestivirus infection and/or -disease.

Surprisingly it was found that this object can be met, and consequently one or more disadvantages of the prior art can be overcome, by providing a mutant Pestivirus that has a chimeric Erns gene which for a large part of the 5′ side is based on an Erns gene from a distantly-related Pestivirus, and the other part of the chimeric Erns gene that is on the 3′ side, is based on an Erns gene from a Pestivirus that is closely-related.

The inventors found that a complete replacement in a Pestivirus of its original Erns gene by a heterologous Erns gene from a Pestivirus that is distantly-related to the receiving Pestivirus, severely reduced or even stopped the replication of the resulting mutant Pestivirus.

This was completely in line with the teaching from the prior art, see e.g. Luo et al. (supra). Also Richter et al. (2011, Virology, vol. 418, p. 113-122) had the same experience with a mutant BVDV with an Erns gene from a Bungowannah virus. Richter et al. tried to overcome this effect on the replication, and made modifications to the signal peptidase cleavage site of the inserted heterologous Erns gene (making a bi-cistronic construct, or a deletion of one nucleotide at the cleavage site). However none of these modifications could restore the viability of the mutant BVDV. It was in no way clear why this effect occurred, and if or how this could be overcome.

The inventors surprisingly found a way to restore the replicative ability of a mutant Pestivirus comprising an Erns gene from a Pestivirus that is genetically distant from the mutant Pestivirus, by providing the distantly-related Erns gene with the 3′ side of an Erns gene from a Pestivirus that is genetically closely related to the mutant Pestivirus.

The 5′ part of the chimeric Erns gene that is from the 5′ side of an Erns gene from a Pestivirus that is genetically distant from the mutant Pestivirus, makes the mutant Pestivirus almost serologically undetectable when using antisera against the Erns protein from a Pestivirus that is genetically distant from the mutant Pestivirus. This provides an excellent serologic marker functionality, with very low risk of misleading cross-reactivity, e.g. when screening a vaccinated animal for infection with a wild type Pestivirus.

In addition, the 3′ part of the chimeric Erns gene that is from the 3′ side of an Erns gene from a Pestivirus that is genetically close to the mutant Pestivirus, makes that the mutant Pestivirus is able to replicate almost at the level it would have without a mutation to its Erns gene. This is important for allowing the mutant Pestivirus to replicate to sufficiently high titres both when the virus is amplified for production purposes, as well as for replicating in the animal, when applied as a live vaccine.

Highly relevant was also the finding that this restoration of replicative capacity by exchange at the 3′ side of the mutated Erns gene did not interfere with or reverse the strong reduction of serologic cross-reaction on Erns protein, that was obtained by the exchanging at the 5′ part of the mutated Erns gene.

Consequently, the present invention allows making and using a mutant Pestivirus with an unexpected and advantageous combination of features: very effective immuno-protective and serologic marker capabilities, with hardly diminished replicative capacity.

It is currently not known why this replacement at the 3′ side of the Erns gene from a Pestivirus that is genetically close to the mutant Pestivirus restores the replication of the mutant Pestivirus. Although the inventors do not want to be bound by any theory or model that might explain these observations, they speculate that apparently the ‘distantly-related’ Erns gene as such does not provide or induce, or at least not sufficiently, certain functions or effects that are important to the viability and replication of the mutant Pestivirus. Only by providing the 3′ side of an Erns gene from a Pestivirus that is genetically close to the mutant Pestivirus, is the missing functionality restored.

Therefore in one aspect, the invention relates to a mutant Pestivirus having a genome wherein the Erns gene is mutated, characterised in that the mutated Erns gene is a chimeric Erns gene, and the chimeric Erns gene consists of a 5′ part and a 3′ part, wherein the 5′ part represents 60-95% of the chimeric Erns gene, and the 3′ part represents the remainder of the chimeric Erns gene, and wherein said 5′ part consists of the corresponding part of an Erns gene from a Pestivirus that is genetically distant from the mutant Pestivirus, and wherein said 3′ part consists of the corresponding part of an Erns gene from a Pestivirus that is genetically close to the mutant Pestivirus.

A “mutant” virus for the invention is a virus that differs genetically from its parent virus in one or more ways. The same applies to a “mutated” gene. Typically a mutant virus or a mutated gene has been constructed in vitro via genetic manipulation.

The mutation can in principle be made by any suitable technique. The net result of the mutation on the viral genome, may be an insertion, deletion and/or substitution.

Methods to mutate a Pestivirus, for example by replacing the original Erns gene by a heterologous Erns gene, are well known in the art. These will typically involve the use of a full length cDNA copy of the Pestiviral genome; for BVDV this is for example pA/BVDV as described in G. Meyers et al. (1996, J. of Virol., vol. 70, p. 8606-8613), and for CSFV: pA/CSFV, as described by G. Meyers et al. (1989, Virology, vol. 171, p. 555-567).

The cDNA copy allows the manipulation by well-known molecular biological techniques involving cloning, transfection, recombination, selection, and amplification. Subsequently RNA is transcribed in vitro from the resulting mutant Pestivirus construct, which can then be transfected into suitable host cells to generate the first generation replicative virus of the mutant Pestivirus.

These, and other techniques are explained in great detail in standard text-books like: Sambrook & Russell: “Molecular cloning: a laboratory manual” (2001, Cold Spring Harbour Laboratory Press; ISBN: 0879695773); Ausubel et al., in: Current Protocols in Molecular Biology (J. Wiley and Sons Inc., NY, 2003, ISBN: 047150338X); C. Dieffenbach & G. Dveksler: “PCR primers: a laboratory manual” (CSHL Press, ISBN 0879696540); and “PCR protocols”, by: J. Bartlett and D. Stirling (Humana press, ISBN: 0896036421). Detailed methods for the construction of a mutant Pestivirus are also described and exemplified herein.

Therefore, a person skilled in the art will readily be able to apply these techniques, using nothing but routine methods and materials.

For the invention, a “Pestivirus” is well known as a virus belonging to the genus Pestivirus. Such a virus displays the characterising features of its taxonomic group-members such as the morphologic, genomic, and biochemical characteristics, as well as the biological characteristics such as physiologic, immunologic, or pathologic behaviour. As is known in the field, the classification of micro-organisms is based on a combination of such features. The invention therefore also includes Pestiviruses that are sub-classified therefrom in any way, for instance as a subspecies, strain, isolate, genotype, variant, subtype or subgroup and the like.

Samples of Pestiviruses for use in the invention, can of course be isolated from infected animals, but more conveniently they are publicly available from universities or (depositary) institutions.

It will be apparent to a skilled person that while a particular Pestivirus for the present invention may currently be classified in a specific species and genus, such a taxonomic classification can change in time as new insights may lead to reclassification into a new or different taxonomic group. However, this does not change the micro-organism itself, its genetic or antigenic repertoire, or the level of genetic relatedness to other viruses, but only its scientific name or classification. Therefore such re-classified micro-organisms remain within the scope of the invention.

For the invention, the word “gene” is used to indicate a nucleic acid- or genomic region that encodes a specific protein. In the case of Pestiviruses, the genome encodes one large open reading frame (ORF), which is translated into a polyprotein. In this case, a gene for one specific protein of that polyprotein, thus does not equal an ORF, and does not have its own promoter, start codon, and stop codon.

An “Erns gene” is readily identifiable by its biological properties. For instance, it is located in the first quarter (the 5′ 25%) of a Pestiviral genome; directly downstream (to the 3′ side) of the gene for the Core protein, and directly upstream (to the 5′ side) of the E1 protein gene. Erns protein is present in the Pestivirus virion and has RNAse activity. A significant portion of the Erns protein is secreted into the environment, and can therefore be detected in the viral culture's medium or the animal host's serum.

The encoded Erns protein in the Pestiviral polyprotein is flanked on both sides by characteristic signal peptides and cleavage sites, and (in currently known species) is between 210 and 227 amino acids long.

By comparative alignments an Erns gene or protein is readily recognised, especially because many nucleotide- and amino acid sequences of Pestivirus Erns genes and proteins are available from public databases. For example the Erns gene of a particular Pestivirus species or atypical isolate for use in the invention can be the Erns gene in the published genome sequence of that Pestivirus in GenBank: BVDV-1: U63479; BVDV-2: U18059; CSFV: X87939; border disease virus: AF037405; HoBi: AB871953; Giraffe: NC003678; Reindeer: AF144618; Antelope: NC024018; Bungowannah: NC023176; NrPV: NC025677; APPV: KR011347; and RaPV: JQ814854 (partial genome, section from E1-NS3).

For illustration: a BVDV Erns gene for use in the invention can be the Erns gene from BVDV-1 strain CP7, of which the viral genomic sequence is available from GenBank acc. nr. U63479, from nucleotide 1179 up to and including 1859, which is 681 nucleotides long. This is represented in SEQ ID NO: 1. This gene encodes the BVDV-1 CP7 Erns protein of 227 amino acids, which is represented in SEQ ID NO: 2.

Similarly: as Bungowannah Erns gene can be used the Erns gene as represented by nucleotides 1228-1893 from GenBank acc.nr. NCO23176, which is 666 nucleotides long, and is presented in SEQ ID NO: 3. The encoded Bungowannah Erns protein is presented in SEQ ID NO: 4. NB: SEQ ID NO's 3 and 4 herein are identical to respectively SEQ ID NO's 6 and 18 of WO2007/121.522.

In the sequence identifiers presented herewith, nucleotides are represented in standard IUPAC-IUB code of DNA. However, as the skilled person will understand, the Pestivirus genomic sequences in nature are in RNA form, where a T will be a U.

A gene is “chimeric” if it is an assembly of parts that were not originally connected. For example an assembly of parts of the same gene from different virus isolates or species. A chimeric gene encodes a chimeric protein, that is effectively a fusion protein.

The terms “5′ part” and “3′ part” are used to indicate the two sections that together constitute the chimeric Erns gene as defined herein. Evidently the 5′ part is located at the 5′ (upstream) side of the chimeric Erns gene, and starts at the first nucleotide of the chimeric Erns gene; the 3′ part is located at the 3′ (downstream) side of the chimeric Erns gene, and ends with the last nucleotide of that gene.

The term “part” as used herein does of itself not imply a certain size, or a division of size. On the contrary: for the present invention the “5′ part” covers a larger section of the chimeric Erns gene than the “3′ part”, as is defined herein:

“the 5′ part represents 60-95% of the chimeric Erns gene”, and

“the 3′ part represents the remainder of the chimeric Erns gene”

Both are simply calculated over the full length of the chimeric Erns gene, whereby it will be evident to a skilled person, that the “remainder” means: the balance of the chimeric Erns gene that is not in the 5′ part. In practice the 3′ part thus represents 5-40% of the chimeric Erns gene at its 3′ side, and is dependant of the size of the 5′ part.

As the skilled person will appreciate, the total nucleotide length of the chimeric Erns gene needs to be such that it is a multiple of three, so as not to introduce a shift in the reading frame of the resulting mutant Pestivirus.

A skilled person is perfectly capable of calculating and optimising the length of the 5′ or 3′ parts of the chimeric Erns gene, on a case-by-case basis, to accommodate this requirement, using nothing but routine methods and materials, and still operate within the scope of the present invention.

As described, the two parts that together form the chimeric Erns gene as defined herein, have a different origin, which results in their different functions. Central in that respect is the level of genetic relatedness of the Pestiviruses from which these parts are derived, and the mutant Pestivirus according to the invention. The basis for this assessment of genetic relatedness is the Erns gene. Compared are: on the one hand the Erns gene from the donor Pestivirus of the 5′- and the 3′ parts of the chimeric Erns gene, and on the other hand the Erns gene that was in the mutant Pestivirus before it was mutated for the invention, i.e. in the parent Pestivirus that was used to create the mutant Pestivirus according to the invention.

So, whether a Pestivirus is “genetically distant from” or “genetically close to” the mutant Pestivirus according to the invention, is to be determined on the basis of the level of nucleotide sequence identity between the donor Erns genes of the 5′- and the 3′ part of the chimeric Erns gene, and the original Erns of the mutant Pestivirus according to the invention.

For ease of making these comparisons, the original Erns genes for the different Pestiviruses are the Erns genes as published in GenBank, for which the accession numbers are described herein above.

For example, when a mutant Pestivirus according to the invention is a CSFV, than the level of genetic relatedness to another Pestivirus is determined by comparing the CSFV Erns gene of GenBank acc.nr. X87939 with the donor Erns gene from that other Pestivirus.

The genetic relatedness for the invention is determined by nucleotide sequence alignment. Such alignments can conveniently be made with one of the many available computer programs, for example: aligning 2 sequences, or aligning a query sequence against a database, can be done using the publicly available program-suite BLAST™, on the NCBI internet website: http://blast.ncbi.nlm.nih.gov/Blast.cgi, using default parameters. Alternatively, a mulltiple alignment of several sequences can conveniently be done using MEGA (Tamura et al., 2013, Mol. Biol. and Evol., vol. 30, p. 2725-2729).

Because the score of a nucleotide sequence alignment is length-dependent, and the length of the parts of the chimeric Erns gene can vary, therefore the alignments are made by simply aligning the entire chimeric Erns gene against the entire original Erns genes as defined above, and then identifying which part of the chimeric Erns gene is from which species or isolate of Pestivirus, and identifying what the length of that part is.

Consequently, after having identified of which Pestivirus the parts of the chimeric Erns gene are derived from, and having identified the length of the parts, then it is determined for the invention, that an Erns gene is from a Pestivirus that is genetically distant from the mutant Pestivirus according to the invention, when the nucleotide sequence identity between the part of that Erns gene as used in the chimeric Erns gene and the corresponding original Erns gene of the mutant Pestivirus is less than 70%, using the program ‘BI2seq’ with default parameters.

Conversely, for the invention, an Erns gene is from a Pestivirus that is “genetically close” to the original Erns gene of the mutant Pestivirus according to the invention, when the nucleotide sequence identity between that part of the Erns gene and the corresponding original Erns gene of the mutant Pestivirus is 70% or more, using the program ‘BI2seq’ with default parameters.

In practice this means that Pestiviruses BVDV-1, BVDV-2, CSFV, border disease virus, Reindeer-, Giraffe-, and HoBi Pestiviruses are ‘genetically close’ to each other, and each of these is ‘genetically distant’ to the Erns gene from Pestiviruses from Antelope, Bungowannah, NRPV, APPV, and RaPV.

This is also illustrated by the dendrogram of FIG. 1, panel C, on page 2997 of Hause et al. (supra).

A “corresponding part” means that the 5′ or the 3′ part of the chimeric Erns gene is formed by a part of similar size and location in the Erns gene of origin. For example: when in an embodiment of a chimeric Erns gene as defined herein, the 5′ part represents 85% of the chimeric Erns gene, than the corresponding part is the 85% at the 5′ side of an Erns gene from a Pestivirus that is genetically distant from the mutant Pestivirus according to the invention.

A similar reasoning applies to the 3′ part of the chimeric Erns gene: when the 5′ part is e.g. 85%, then the 3′ part represents the ‘remaining’ 15% of the chimeric Erns gene, and than the ‘corresponding part’ is the 15% at the 3′ side of an Erns gene from a Pestivirus that is genetically close to the mutant Pestivirus according to the invention.

In an embodiment, an Erns gene is from a Pestivirus that is genetically distant from the mutant Pestivirus according to the invention, when the nucleotide sequence identity between the part of that Erns gene as used in the chimeric Erns gene and the corresponding original Erns gene of the mutant Pestivirus is less than 65% using the program ‘BI2seq’ with default parameters.

Preferably, genetically distant means less than 60%, 55, or even 50% nucleotide sequence identity between the part of that Erns gene and the corresponding original Erns gene of the mutant Pestivirus, using the program ‘Bl2seq’ with default parameters, in this order of preference.

Conversely, in an embodiment, an Erns gene is from a Pestivirus that is genetically close to the mutant Pestivirus according to the invention, when the nucleotide sequence identity between the part of that Erns as used in the chimeric Erns gene and the corresponding original Erns gene of the mutant Pestivirus is more than 75% upon alignment using the program ‘BI2seq’ with default parameters.

Preferably, genetically close means more than 80%, 85%, or even more than 90% nucleotide sequence identity between the part of that Erns gene in the chimeric Erns gene and the corresponding original Erns gene of the mutant Pestivirus, using the program ‘BI2seq’ with default parameters, in this order of preference.

In an embodiment, the 5′ part of the chimeric Erns gene is between about 65 and about 93% of the chimeric Erns gene. Preferably between about 70 and about 93%; between about 75 and about 91%; or even between about 80 and about 90% of the chimeric Erns gene, in that order of preference.

For the invention, a number indicated with the term “about” means that number can vary between ±25% around the indicated value; preferably about means ±20% around the indicated value, more preferably about means ±15, 12, 10, 8, 6, 5, 4, 3, 2% around the indicated value, or even about means ±1% around the indicated value, in that order of preference.

Within the currently known members of the genus Pestivirus, BVDV, CSFV, and border disease virus have the greatest economic impact on the agricultural sector.

Therefore in an embodiment, a mutant Pestivirus according to the invention is a Pestivirus selected from the group consisting of: bovine viral diarrhoea virus (BVDV); classical swine fever virus (CSFV); and border disease virus.

When the mutant Pestivirus according to the invention is based upon BVDV, CSFV, or border disease virus, then an Erns gene from a Pestivirus that is genetically distant, is an Erns gene from Antelope Pestivirus, Bungowannah virus, Norway rat Pestivirus, APPV, or Rhinolophus affinis Pestivirus.

Therefore in an embodiment of a mutant Pestivirus according to the invention, the Erns gene from a Pestivirus that is genetically distant, is an Erns gene from a Pestivirus selected from the group consisting of: Antelope Pestivirus; Bungowannah virus; Norway rat Pestivirus; atypical porcine Pestivirus (APPV); and Rhinolophus affinis Pestivirus (RaPV).

Further, when the mutant Pestivirus according to the invention is based upon BVDV, CSFV, or border disease virus, then an Erns gene from a Pestivirus that is genetically close, is an Erns gene from BVDV-1, BVDV-2, CSFV, border disease virus, Reindeer Pestivirus, Giraffe Pestivirus, or HoBi Pestivirus.

Therefore in an embodiment of a mutant Pestivirus according to the invention, the Erns gene from a Pestivirus that is genetically close, is an Erns gene from a Pestivirus selected from the group consisting of: BVDV-1; BVDV-2; CSFV; border disease virus; Reindeer Pestivirus; Giraffe Pestivirus; and HoBi Pestivirus.

An advantageous use of the mutant Pestivirus according to the present invention is as a marker vaccine. When that marker vaccine is applied as a live vaccine, the mutant Pestivirus needs to have a reduced virulence, in order to be sufficiently safe to administer to animals.

Therefore in an embodiment, a mutant Pestivirus according to the invention is an attenuated Pestivirus.

For the invention, a Pestivirus is “attenuated” if the virus is having a reduced virulence as compared to another virus of the same species or isolate, such as a wild type isolate. In fact attenuated means to display a reduced dissemination through the body of an infected target animal, e.g. foetal infection; to induce less pathology such as (signs of) disease; and/or to display a reduced spread into the environment.

Whether a Pestivirus is actually attenuated, and if that level of attenuation is sufficient for use as the parent virus for a mutated Pestivirus according to the invention, e.g. regarding its use in a life vaccine, can conveniently be determined using standard procedures either in vitro or in vivo. For example by comparing side by side two variants of a Pestivirus, one with and one without that mutation. For example, by comparing the effect of the mutation on the viral replication rate in cell culture, or in an experimentally infected animal: checking viral presence in different tissues or organs, and monitoring clinical, macroscopic, or microscopic signs of disease in an animal or a foetus.

One way to obtain that attenuation is by providing the mutant Pestivirus with a further mutation that attenuates its virulence to acceptable levels for use as a live attenuated vaccine.

Examples of further mutations that can attenuate a mutant Pestivirus according to the invention, are mutations in the Npro- or in the NS3 genes.

A mutation in NS3 is preferably a mutation as described in WO 2014/033.149, whereby a Pestivirus has a mutation of an epitope located in a helicase domain of NS3 protein, so that the epitope is no longer reactive with a monoclonal antibody against that epitope in a wild-type Pestivirus.

Alternatively, or in addition, the further mutation is located in the Npro gene. Such a mutation can provide a level of attenuation that combines well with the other modifications in the mutant Pestivirus according to the invention.

Therefore in an embodiment, a mutant Pestivirus according to the invention has a genome wherein the Npro gene is mutated.

In a preferred embodiment, the mutation to the Npro gene is a mutation as described in WO 2008/034.857, whereby the Npro gene is deleted, except for the 5′ part of the Npro gene that encodes the N-terminal 12 amino acids of Npro.

Further mutations or attenuations can be made to increase the safety or the efficacy of the mutant Pestivirus when used as a live attenuated marker vaccine.

One particularly useful adaptation is described in WO 2012/038.454. This invention prevents the interference that occurs when BVDV viruses of different genotypes are combined in one vaccine. As described in WO 2012/038.454, a BVDV Pestivirus of one genotype is mutated to comprise an E2 gene of a BVDV of another genotype, instead of its own E2 gene. The effect is that such a E2-chimeric BVDV can then be combined in one vaccine with a BVDV that has the same viral backbone but its original E2 gene. These two viruses now will no longer interfere with the development of an immune response against each one.

Therefore in an embodiment of a mutant Pestivirus according to the invention, the mutant Pestivirus is based upon a BVDV, and said BVDV is of one genotype, but comprises an E2 gene from a BVDV of another genotype, instead of its original E2 gene.

In a preferred embodiment, a mutant Pestivirus according to the invention is based upon a BVDV-1 and comprises an E2 gene of BVDV-2 instead of its original E2 gene.

This adaptation can also be comprised in a mutant Pestivirus according to the invention, e.g. when the mutant Pestivirus is based upon a BVDV-1 virus and comprises a BVDV-1 E2 gene. The reverse combination is of course also possible, where the backbone of the mutant Pestivirus according to the invention is based upon a BVDV-2 and comprises a BVDV-2 E2 gene.

In an embodiment, a mutant Pestivirus according to the invention is a BVDV, and said BVDV is of the cytopathogenic biotype.

In an embodiment of the mutant Pestivirus according to the invention, the chimeric Erns gene comprises as the Erns gene from a Pestivirus that is genetically distant, the Erns gene from a Bungowannah virus.

Such a mutant Pestivirus was found to have excellent marker functionality, because the encoded chimeric Erns protein was found to be only detectable using anti-Bungowannah virus antisera, but not when using antisera against other Pestiviruses or against their Erns protein.

In a preferred embodiment, the mutant Pestivirus according to the invention is based on BVDV, and comprises a chimeric Erns gene comprising a Bungowannah Erns gene as the Erns gene from a Pestivirus that is genetically distant, and the 3′ part of the chimeric Erns gene is about 10 and about 20% of the chimeric Erns, and is derived from a BVDV.

In a preferred embodiment of the mutant Pestivirus according to the invention, the mutant Pestivirus is based on BVDV-1 and comprises a chimeric Erns gene as described in SEQ ID NO: 5.

The chimeric Erns gene of SEQ ID NO: 5 is 687 nucleotides long, and has the correct reading frame. In this case the level of relatedness between this 3′ part of the chimeric Erns gene and the mutant Pestivirus, here BVDV-1, is thus 100%, which qualifies as genetically close.

Regarding the length percentage of this 3′ part of this chimeric Erns gene as defined herein, that is 108 nucleotides from the (corresponding) 3′ side of the Erns gene of a BVDV-1, strain CP7 (original Erns gene is 681 nt long); therefore in this chimeric Erns gene the 3′ part as defined herein is (108/687)=15.7% of this chimeric Erns gene.

SEQ ID NO: 6 presents the amino acid sequence of the chimeric Erns gene encoded by SEQ ID NO: 5.

The 5′ part of the Bungowannah Erns gene (SEQ ID NO: 3) that is in SEQ ID NO: 5 has 65% nucleotide sequence identity with the corresponding length (579 nucleotides) of the original Erns gene, here: the BVDV-1 Erns gene of acc. nr. U63479. This qualifies as genetically distant.

The construction and use of such a mutant Pestivirus is described in detail in the Example section hereinafter.

In a further aspect the invention relates to a chimeric Erns gene as defined in the invention.

A chimeric Erns gene can be used to construct a mutant Pestivirus according to the invention. The gene may be comprised in a PCR amplificate, or in a plasmid or other vehicle to facilitate modification and cloning. The gene or the plasmid can be amplified by PCR or in a bacterial culture, using standard molecular-biological techniques.

In a preferred embodiment the chimeric Erns gene is as presented in SEQ ID NO: 5.

Further or additional adaptations or mutations of the mutant Pestivirus according to the invention are conceivable. Also these may be applied in one or more combination(s). Therefore in an embodiment of a mutant Pestivirus according to the invention, one, more, or all of the conditions apply, selected from the group consisting of:
the mutant Pestivirus comprises a further mutation, which is located in the Npro gene, and which attenuates the mutant Pestivirus,
the mutant Pestivirus is based upon a BVDV of one genotype, but comprises an E2 gene from a BVDV of another genotype, instead of its original E2 gene,
the Erns gene from a Pestivirus that is genetically distant, is an Erns gene from a Bungowannah virus,
the Erns gene from a Pestivirus that is genetically close, is an Erns gene from a BVDV-1 or a BVDV-2,
the mutant Pestivirus is based upon a cp biotype BVDV-1 or upon a cp biotype BVDV-2, and
the chimeric Erns gene is as represented in SEQ ID NO: 5.

In the construction of the mutant Pestivirus according to the invention, there are different ways in which the various embodiments can be introduced. For example a mutant Pestivirus according to the invention can be generated by the introduction of a chimeric Erns gene as defined herein. Next further mutations and variations can be added to the mutant Pestivirus.

However, a more favourable approach may be to introduce the chimeric Erns gene as defined herein into a Pestivirus that already has one or more of the other embodiments, and in this way generate a mutant Pestivirus according to the invention, having several additional features. For example this may be applied to a Pestivirus that is an established vaccine strain. In this way that established vaccine can be provided with efficient marker properties, while maintaining its replication and immuno-protection.

Therefore in a further aspect the invention provides a method for the construction of a mutant Pestivirus according to the invention, said method comprising mutating the Erns gene in a Pestivirus genome into a chimeric Erns gene as defined herein.

The methods and materials required for the application of the method according to the invention are well within the routine capabilities of the skilled person, are described in detail herein, and are well known in the art.

In an embodiment the method according to the invention is applied to a Pestivirus that is used as an established vaccine strain. For example: in an inactivated vaccine such as: for BVDV: Bovilis® BVD (MSD Animal Health), Bovidec® (Novartis), Pregsure® BVD (Zoetis); for border disease and BVDV: Mucobovin® (Merial).

Or preferably, in an established live attenuated Pestivirus vaccine strain, such as: for BVDV: Mucosiffa® (Merial); and for CSFV: Porcilis CSF Live (MSD AH); Suvaxyn CSF (Zoetis); or Riemser Schweinepest vakzine [Swine fever vaccine of Riems] (IDT Dessau).

In the method according to the invention, and for the amplification of the mutant Pestivirus according to the invention, the virus is produced in suitable host cells. This may be by way of a transfection of nucleic acid into such a host cell, when the mutant virus is not yet in a replicative form. Alternatively, when in a replicative form, the mutant Pestivirus is inoculated onto such host cells and is amplified by natural replication.

The host cell can be a primary cell, such as prepared from an animal tissue. Preferably however the host cell is from an established cell-line, growing continuously.

At certain points in the viral replication cycle, such a host cells will contain a mutant Pestivirus according to the invention.

Therefore in a further aspect, the invention relates to a host cell comprising a mutant Pestivirus according to the invention, or as obtainable by a method according to the invention.

Suitable host cells for the replication of Pestiviruses are well known in the art, and are generally publicly available, e.g. from universities or (depositary) institutions. Methods, media, and materials for preparing and culturing a host cell according to the invention, are well known in the art.

Examples of suitable host cells are cell lines such as: bovine cell lines such as: MDBK (Madin Darby bovine kidney); swine cell lines such as: PK15 (porcine kidney), or STE (swine testicular epitheloid); or general-purpose cell lines such as: Vero (African green monkey kidney cells), MDCK (Madin Darby canine kidney), or PT cells (ovine epithelial kidney cells).

As discussed above, an advantageous use of a mutant Pestivirus according to the invention, is as a marker vaccine.

Therefore in a further aspect, the invention relates to a vaccine for animals comprising a mutant Pestivirus according to the invention, or a host cell according to the invention, or any combination thereof, and a pharmaceutically acceptable carrier.

A “vaccine” is well known to be a composition that has an inherent medical effect. A vaccine comprises an immunologically active component, and a pharmaceutically acceptable carrier. The ‘immunologically active component’, is one or more antigenic molecule(s) that is recognised by the immune system of a target, here: the mutant Pestivirus according to the invention, and that induces a protective immunological response. The response may originate from the targets' innate- and/or from the acquired immune system, and may be of the cellular- and/or of the humoral type.

A vaccine generally is efficacious in reducing the level or the extent of an infection, for example by reducing the viral load or shortening the duration of viral replication in a host animal.

Also, or possibly as a results thereof, a vaccine generally is effective in reducing or ameliorating the symptoms of disease that may be caused by, or may the result of, such viral infection or replication, or by the animal's response to that infection.

The effect of the vaccine according to the invention is the prevention or reduction in animals of an infection by a Pestivirus and/or of signs of disease that are associated with such virus infection or replication, through the induction of an immunological response, such as the induction of virus-neutralising antibodies, and/or the induction of a cellular immune response.

Such a vaccine may colloquially be referred to as a vaccine ‘against’ a Pestivirus, or as a ‘Pestivirus vaccine’.

Determining the effectiveness of a vaccine against Pestivirus can e.g. be done by monitoring the immunological response following vaccination or after a challenge infection, e.g. by monitoring the targets' signs of disease, clinical scores, serological parameters, or by re-isolation of the pathogen, and comparing these results to a vaccination-challenge response seen in mock-vaccinated animals.

Alternatively, in cases where virus neutralising antibodies, above certain levels, are known to be correlated to protection, serology can suffice to demonstrate vaccine efficacy.

The “animals” for which the vaccine according to the invention is intended are animals that are susceptible to infection with a Pestivirus. Mainly these will be mammalian (non-human) animals, and will be members of the order Artiodactyla. Preferred target animals for the vaccine according to the invention are ruminants and swine; more preferred are: cattle, sheep, and swine.

The vaccine according to the invention may comprise any “combination” of the mutant Pestivirus and the host cell, both according to the invention. This refers to the variety of ways a vaccine can be prepared, as is described below. One example is the harvesting of a complete culture of a mutant Pestivirus according to the invention, including both the virus and the (infected) host cells.

A vaccine according to the invention may be a life-, an inactivated-, or a subunit vaccine, or any combination thereof.

An ‘inactivated’ vaccine is a vaccine comprising a micro-organism that has been rendered non-replicative by some method of inactivation. Common methods of inactivation are by applying e.g. heat, radiation, or chemicals such as formalin, beta-propiolactone, binary ethyleneimine, or beta-ethanolamine.

The mutant Pestivirus to be inactivated initially is a whole virus particle that can be derived from a viral culture, such as from the cell-pellet, the culture supernatant, or the whole culture. As the inactivation method affects the proteins, the lipids, and/or the nucleic acids of the virus particle, this may to some extend become damaged. Nevertheless this type of vaccine is commonly called a whole virus inactivated vaccine.

The selection of a suitable method of inactivation, is well within the routine capabilities of the person skilled in the art.

Alternatively, a vaccine according to the invention, or a part thereof, may be a subunit vaccine. This can be prepared either from live- or from inactivated virus, by applying one or more (additional) steps for the fractionation or isolation of one or more parts of the viral particle. This comprises for instance preparing an extract, fraction, homogenate, or sonicate, all well known in the art.

However the preferred form of a vaccine according to the invention is a live vaccine. Although the term ‘live’ is biologically incorrect in respect of a viral agent, it is commonly used in this field. Consequently, for the invention the term ‘live’ refers to a mutant Pestivirus according to the invention that is capable of replication, i.e. is replicative, of non-inactivated.

The vaccine according to the invention can advantageously be used as a marker vaccine for Pestivirus Erns protein, because of the properties of the mutant Pestivirus according to the invention, in combination with screening via appropriate tests.

In a preferred embodiment, the vaccine according to the invention is a live attenuated marker vaccine.

Live attenuated vaccines are commonly prepared in freeze-dried form. This allows prolonged storage at temperatures above freezing. Procedures for freeze-drying are known to persons skilled in the art, and equipment for freeze-drying at a variety of scales is available commercially.

Therefore, in an embodiment, the vaccine according to the invention is in a freeze-dried form.

The freeze-dried form can be a cake, or can be a lyosphere, or both as described in EP 799.613.

To reconstitute a freeze-dried vaccine, it is suspended in a physiologically acceptable diluent. This is commonly done immediately before administration, to ascertain the best quality of the vaccine. The diluent is typically aqueous, and can e.g. be sterile water, or a physiological salt solution. The diluent to be used for reconstituting the vaccine can itself contain additional compounds, such as an adjuvant.

In a further embodiment of the freeze dried vaccine according to the invention, the diluent for the vaccine is supplied separately from the freeze-dried form comprising the active vaccine composition. In that case, the freeze-dried vaccine and the diluent composition form a kit of parts that together embody the vaccine according to the invention.

Therefore, in a preferred embodiment of the freeze-dried vaccine according to the invention, the vaccine is a kit of parts with at least two containers, one container comprising the freeze-dried vaccine, and one container comprising an aqueous diluent.

A “pharmaceutically acceptable carrier” is for example a liquid such as water, physiological salt solution, or phosphate buffered saline solutions. In a more complex form the carrier can e.g. be a buffer comprising further additives, such as stabilisers or preservatives.

A vaccine according to the invention may also comprise an adjuvant. This is particularly useful when the vaccine is an inactivated- or a subunit vaccine. However, also live vaccines can comprise an adjuvant, although that should be carefully selected not to reduce the viability of the vaccine virus, even upon prolonged storage.

An “adjuvant” is a well-known vaccine ingredient, which in general is a substance that stimulates the immune response of a target in a non-specific manner. Many different adjuvants are known in the art. Examples of adjuvants for inactivated/subunit vaccines are: Freund's Complete or -Incomplete adjuvants, vitamin E, aluminium compositions such as Aluminium-phosphate or Aluminium-hydroxide, Polygen™, non-ionic block polymers and polyamines such as dextran sulphate, Carbopol™, pyran, Saponin, such as: Quil A™, or Q-vac™. Saponin and vaccine components may be combined in an ISCOM™.

Furthermore, peptides such as muramyldipeptides, dimethylglycine, tuftsin, are often used as adjuvant, and oil-emulsions, using mineral oil e.g. Bayol™ or Markol™, Montanide™ or light mineral (paraffin) oil; or non-mineral oil such as squalene, squalane, or vegetable oils, e.g. ethyl-oleate. In addition, combination products such as ISA™ (from Seppic) or DiluvacForte™ can advantageously be used.

A vaccine-emulsion can be in the form of a water-in-oil (w/o), oil-in-water (o/w), water-in-oil-in-water (w/o/w), or a double oil-emulsion (DOE), etc.

Alternatively, and more suitable for use with a live vaccine: other immuno-stimulatory components may be added to the vaccine according to the invention, such as a cytokine or an immunostimulatory oligodeoxynucleotide.

The immunostimulatory oligodeoxynucleotide is preferably an immunostimulatory non-methylated CpG-containing oligodeoxynucleotide (INO). A preferred INO is a Toll-like receptor (TLR) 9 agonist, such as described in WO 2012/089.800 (X4 family), WO 2012/160.183 (X43 family), or WO 2012/160.184 (X23 family).

A vaccine according to the invention should be administered to the target animals in an optimal way in respect of its dose, volume, route, or formulation, as well in an optimal way with respect to the target animal's age, sex, or health status. The skilled person is perfectly capable of determining such optimal conditions for the vaccine administration. For an inactivated or subunit Pestivirus vaccines, the administration will typically be by intra-muscular, subcutaneous, or intradermal injection. For a live attenuated vaccine according to the invention, a ‘mucosa!’ route may also be appropriate, such as intra-nasal, or ocular.

Therefore, in an embodiment the vaccine according to the invention is administered by parenteral route. Preferably by intramuscular, subcutaneous, or intradermal route.

A live vaccine according to the invention can also be administered by injection. Alternatively, and depending on the specific properties of the mutant Pestivirus employed, it may be applied via a mucosal, oral, or respiratory route.

In an embodiment the vaccine according to the invention is administered by mucosal route. Preferably by intra-nasal, or ocular route.

Preferably the live vaccine is applied via a method of mass application, such as by spray, or via the feed or the drinking water.

A vaccine according to the invention can advantageously be combined with another antigen, micro-organism or vaccine component, into a combination vaccine. Depending on the characteristics of the particular form of vaccine according to the invention, the way to make that combination needs to be carefully selected. Such choices are within the routine capabilities of the skilled person.

Therefore, in an embodiment, a vaccine according to the invention is characterised in that it comprises at least one additional immunoactive component.

An “additional immunoactive component” may be an antigen, an immune enhancing substance, and/or a vaccine, either of which may comprise an adjuvant. The additional immunoactive component when in the form of an antigen may consist of any antigenic component of veterinary importance. Preferably the additional immunoactive component is based upon, or derived from, a further micro-organism that is pathogenic to the target animal. It may for instance comprise a biological or synthetic molecule such as a protein, a carbohydrate, a lipopolysaccharide, a nucleic acid encoding a proteinaceous antigen. Also a host cell comprising such a nucleic acid, or a live recombinant carrier micro-organism containing such a nucleic acid, may be a way to deliver or express the nucleic acid or the additional immunoactive component. Alternatively the additional immunoactive component may comprise a fractionated or killed micro-organism such as a parasite, bacterium or virus.

The additional immunoactive component(s) may also be an immune-enhancing substance e.g. a chemokine, or an immunostimulatory nucleic acid as described above. Alternatively, the vaccine according to the invention, may itself be added to a vaccine.

An advantageous utility of a combination vaccine for the invention is that it not only induces an immune response against Pestivirus, but also against other pathogens of a target animal, while only a single handling of the animal for the vaccination is required, thereby reducing discomfort to the animal, as well as time- and labour costs.

Examples of such additional immunoactive components are in principle all viral, bacterial, and parasitic pathogens, or parts thereof, that are amenable to vaccination of an animal that is also a target for a Pestivirus vaccine according to the invention.

Examples of such pathogens relevant for target animals are:

For swine: porcine circovirus, porcine reproductive and respiratory syndrome virus, pseudorabies virus, porcine parvo virus, classical swine fever virus, Mycoplasma hyopneumoniae, Lawsonia intracellularis, E. coli, Streptococcus spec., Salmonella spec., Clostridia spec., Actinobacillus pleuropneumoniae, Pasteurella spec., Haemophilus spec., Erysipelothrix spec., and Bordetella spec.

For cattle: Neospora spec., Dictyocaulus spec., Cryptosporidium spec., Ostertagia spec., bovine rotavirus, bovine viral diarrhoea virus, bovine coronavirus, infectious bovine rhinotracheitis virus (bovine herpes virus), bovine paramyxovirus, bovine parainfluenza virus, bovine respiratory syncytial virus, rabies virus, bluetongue virus, Pasteurella haemolytica, E. coli, Salmonella spec., Staphylococcus spec., Mycobacterium spec., Brucella spec., Clostridia spec., Mannheimia spec., Haemophilus spec., and Fusobacterium spec.

For sheep: Toxoplasma gondii, peste des petit ruminant virus, bluetongue virus, Schmallenberg virus, Mycobacterium spec., Brucella spec., Clostridia spec., Coxiella spec., E. coli, Chlamydia spec., Clostridia spec., Pasteurella spec., and Mannheimia spec.

The additional immunoactive component may thus also be a further Pestivirus, and/or a Pestivirus vaccine, either or both of which may be live, inactivated, or a subunit vaccine.

A skilled person is more than capable of making such combinations, while safeguarding the efficacy, safety and stability of the vaccine according to the invention.

The manufacture of a vaccine according to the invention is well known in the art, and is within the routine capabilities of the skilled person. Such methods of manufacture will in general comprise steps for the propagation of a mutant Pestivirus according to the invention, e.g. in an in vitro cell-culture, harvesting, and formulation depending on the type of vaccine to be prepared.

Therefore in a further aspect, the invention relates to a method for the preparation of a vaccine according to the invention, the method comprising the steps of:

infecting a culture of host cells with a mutant Pestivirus according to the invention,

incubating the infected culture of host cells,

harvesting the culture or a part thereof, and

admixing the culture or the part thereof, with a pharmaceutically acceptable carrier.

At different points in this method, additional steps may be added, for example for additional treatments such as for purification or storage.

Next, the method of preparation can involve the admixing with further pharmaceutically acceptable excipients such as stabilisers, carriers, adjuvants, diluents, emulsions, and the like. The prepared vaccine is then apportioned into appropriate sized containers. The various stages of the manufacturing process will be monitored by adequate tests, for instance by immunological tests for the quality and quantity of the antigens; by microbiological tests for inactivation (if applicable), sterility, and absence of extraneous agents; and ultimately by in vitro or in vivo experiments to determine vaccine efficacy and -safety. All these are well known to a skilled person, and are prescribed in Governmental regulations such as the Pharmacopoeia, and in handbooks such as “Remington: the science and practice of pharmacy” (2000, Lippincot, USA, ISBN: 683306472), and: “Veterinary vaccinology” (P. Pastoret et al. ed., 1997, Elsevier, Amsterdam, ISBN 0444819681).

A vaccine for the invention is manufactured into a form that is suitable for administration to an animal target, and that matches with the desired route of application, and with the desired effect.

The vaccine can be formulated as an injectable liquid, such as: a suspension, solution, dispersion, or emulsion. Alternatively the vaccine can be formulated in a freeze-dried form. Commonly vaccines are prepared sterile.

Depending on the route of application of the vaccine according to the invention, it may be necessary to adapt the vaccine's composition. This is well within the capabilities of a skilled person, and generally involves the fine-tuning of the efficacy, stability, or safety of the vaccine. This can be done by adapting the vaccine dose, quantity, frequency, route, by using the vaccine in another form or formulation, or by adapting the other constituents of the vaccine (e.g. a stabiliser or an adjuvant).

The exact amount of mutant Pestivirus according to the invention to be used per animal dose of the vaccine according to the invention, depends on the type of the vaccine and on the target animal treated. For a live vaccine this is typically less than for an inactivated vaccine as the live virus can replicate. As an indication, a dose of live vaccine according to the invention will contain between about 1×10̂1 and about 1×10̂7 tissue culture infective dose 50% (TCID50)/animal of the mutant Pestivirus according to the invention. A dose of inactivated vaccine according to the invention will contain the pendant of between about 1×10̂2 and about 1×10̂9 TCID50/animal of mutant Pestivirus according to the invention, in inactivated form.

Methods to count and quantify viral particles of the mutant Pestivirus according to the invention are well known.

The volume per animal dose of the vaccine according to the invention can be optimised according to the target animal for which the treatment is intended, and the intended route of application. Typically an inactivated vaccine is given at a dose of between 0.1 and 5 ml/animal. The dose of a live vaccine is even more variable dependent on the route applied.

The determination of what is an immunologically effective amount of the vaccine according to the invention, or the optimisation of the vaccine's volume per dose, are both well within the capabilities of the skilled artisan.

In a further aspect the invention relates to a mutant Pestivirus according to the invention, or to a host cell according to the invention, or to any combination thereof, for use in a vaccine for animals.

In a further aspect the invention relates to a use of a mutant Pestivirus according to the invention, or of a host cell according to the invention, or of any combination thereof, for the manufacture of a vaccine for animals.

In a further aspect the invention relates to a use of a vaccine according to the invention, for the prevention or reduction of an infection by a Pestivirus or of associated signs of disease in animals.

In a further aspect the invention relates to a method for the prevention or reduction of an infection by a Pestivirus or of associated signs of disease in animals, the method comprising the administration of a vaccine according to the invention to said animals.

In a further aspect the invention relates to a method of vaccination of animals, comprising the step of administering to said animals a vaccine according to the invention.

A vaccine according to the invention can thus be used either as a prophylactic- or as a therapeutic treatment, or both, as it interferes both with the establishment and with the progression of an infection by a Pestivirus.

In that respect, a further advantageous effect of the reduction of viral load by the vaccine according to the invention, is the prevention or reduction of shedding and thereby the spread of the virus, both vertically to offspring, and horizontally within a herd or population, and within a geographical area. Consequently, the use of a vaccine according to the invention leads to a reduction of the prevalence of a Pestivirus.

Therefore further aspects of the invention are:
the use of a vaccine according to the invention for reducing the prevalence of a Pestivirus in a population or in a geographical area, and
the vaccine according to the invention for reducing the prevalence of a Pestivirus in a population or in a geographical area.

The administration regime for applying the vaccine according to the invention to a target organism can be in single or in multiple doses, in a manner compatible with the formulation of the vaccine, with practical aspects of the animal husbandry, and in such an amount as will be immunologically effective.

Preferably, the regimen for the administration of a vaccine according to the invention is integrated into existing vaccination schedules of other vaccines that the target animal may require, in order to reduce stress to the animals and to reduce labour costs. These other vaccines can be administered in a simultaneous, concurrent or sequential fashion, in a manner compatible with their registered use.

It is advantageous to apply the vaccine according to the invention as early as it is possible to establish an effective immune protection in the target animal. This will also incorporate the relevance and the level of maternally derived antibodies in the target.

As discussed above, the mutant Pestivirus and the vaccine, both according to the invention, are particularly suitable in a protocol applying the DIVA principle. This because the mutant Pestivirus provides the vaccine with powerful marker vaccine properties. This applies both when the vaccine is a life—as when it is an inactivated—or subunit vaccine.

The vaccine according to the invention induces in a vaccinated target animal, antibodies against an Erns protein that are not readily able to bind specifically with an Erns protein of a Pestivirus that is different from the vaccine virus. This allows several ways of devising screening assays:

On the one hand an assay can be devised for specifically detecting the mutant Pestivirus according to the invention, as a positive marker, screening for effective vaccination. Such an assay would use antibodies against the Erns protein expressed by the mutant Pestivirus according to the invention, or would use the mutant Pestivirus or its Erns protein as detection antigen.

On the other hand, an assay can be devised to positively detect Pestiviruses that are different compared to the mutant Pestivirus comprised in a vaccine according to the invention, as negative marker screening. This detection of non-vaccine virus would thus allow screening for infection with any pathogenic wild-type field virus, even in Pestivirus vaccinated animals, thanks to the advantageous marker properties of the vaccine according to the invention. Such an assay would use antibodies against Erns that do not recognise the Erns as expressed by the mutant Pestivirus according to the invention, or use pathogenic virus, or its Erns protein for the detection.

Therefore a further aspect of the invention is a method for differentiating animals vaccinated with a vaccine according to the invention, from animals infected with a Pestivirus other than a mutant Pestivirus comprised in the vaccine, the method comprising the use of an antibody against an Erns protein, which antibody does not bind specifically with the chimeric Erns protein expressed by a mutant Pestivirus that is comprised in the vaccine.

For the invention, “antibodies” are immunoglobulin proteins or parts thereof that can specifically bind to an epitope. For sero-diagnosis, antibodies will typically be of IgG or IgM type. The antibodies can be intact or partial antibodies, e.g. a single chain antibody, or a part of an immunoglobulin containing the antigen-binding region. They can be of a different form: a (synthetic) construct of such parts, provided the antibody-parts still contain an antigen-binding site. Well known sub-fragments of immunoglobulins are: Fab, Fv, scFv, dAb, or Fd fragments, Vh domains, or multimers of such fragments or domains. Also the antibodies can be labelled in one or more ways to facilitate or amplify detection.

Antibodies for use as reagent in diagnostic assays are commonly produced by (over-)immunising a donor animal with the target antigen, and harvesting the antibodies produced from the animal's serum. Well known donors are rabbits and goats. Another example is chickens which can produce high levels of antibodies in the egg-yolk, so-called IgY. Alternatively, antibodies can be produced in vitro, e.g. via the well-known monoclonal antibody technology from immortalized B-lymphocyte cultures (hybridoma cells), and for which industrial scale production systems are known. Also antibodies or fragments thereof may be expressed in a recombinant expression system, through expression of the cloned Ig heavy- and/or light chain genes. All these are well known to the skilled artisan.

As is well known in the art, antibodies directed “against” a certain target, are antibodies that are specific for an epitope on that target, whereby the target is a particular molecule or entity. An antibody (or fragment thereof) is specific for an epitope if it is capable of selective binding to that epitope.

For the invention antibodies will be referred to based on the target against which they are directed; e.g.: antibodies against CSFV are referred to as ‘CSFV antibodies’, and ‘Erns antibodies’, are antibodies against Erns protein a.k.a. anti-Erns antibodies, etc.

Whether an antibody can “bind specifically” to an epitope or not, can easily be assessed by a skilled person. For example, the specificity of results of an inhibition-based immune-assay can be determined by demonstrating the inhibition is correlated with the concentration of the antigen or of the antibody used in the assay. Using e.g. a competition binding assay, it can be determined how much of an antigen is required to inhibit antibody binding to coated antigen by 50% (Bruderer et al., 1990, J. of Imm. Meth., vol. 133, p. 263).

Antibodies against Erns protein that do not recognise the Erns protein of a mutant Pestivirus according to the invention, are known in the art or can be obtained using routine procedures. Described ELISA tests for Pestivirus Erns protein employ anti-Erns antibodies raised against e.g. CSFV or BVDV-1, see e.g. Grego et al. (2007, J. of Vet. Diagn. Invest., vol. 19, p. 21-27). These will not bind specifically with the Erns of the mutant Pestivirus according to the invention.

Consequently, one further advantageous use of the present invention, is that existing tests based on Pestivirus Erns-antibodies can be employed in the methods according to the invention.

The method for differentiating animals according to the invention is particularly relevant to the Pestiviruses of greatest agro-economical relevance.

Therefore in an embodiment of the method for differentiating animals according to the invention, the antibody binds specifically with an Erns protein from a Pestivirus selected from the group consisting of: BVDV-1; BVDV-2; CSFV; and border disease virus.

The method for differentiating according to the invention can be performed using any suitable method of immune-diagnostic assay.

Often such immune-diagnostic assays will have a step for amplifying the signal strength, and one or more steps for washing away unbound, unspecific or unwanted components. The detection of a positive signal can be done in a variety of ways such as optically by detecting a colour change, a fluorescence, or a change in particle size, or alternatively by the detection of radioactively labelled antigens or antibodies in immune-complexes. Similarly, the physical form of the test can vary widely and can e.g. employ a microtitration plate, a membrane, a dipstick, a biosensor chip, a gel matrix, or a solution comprising (micro-) carrier particles such as latex, metal, or polystyrene, etc.

The choice for a particular set-up of such an immune-diagnostic assay is usually determined by the type of input sample, the desired test sensitivity (correctly identifying a positive sample), and test specificity (correctly discriminating between true positive and true negative samples). Such properties are dependent of the strength and timing of an immune response, or the presence of a micro-organism. Further the requirements for test-economy such as the applicability on a large scale and the costs may be decisive for selection of a particular format.

Well-known immuno-diagnostic tests are: radioimmuno assays, immunodiffusion, immunofluorescence, immune-precipitation, agglutination, haemolysis, neutralisation, and “enzyme-linked immuno-sorbent assay” or ELISA. Especially for large scale testing, the automation of the liquid handling, and/or of the result reading and processing, may be a requirement. This may also require replacing a traditional assay by a more modern and miniaturised format such as in AlphaLISA™ (Perkin Elmer).

ELISA's are easily scalable, and can be very sensitive. A further advantage is the dynamic range of its results because samples can be tested in a dilution range. Results are expressed in arbitrary units of absorbance, typically between 0.1 and 2.5 optical density (OD) units, or as ‘blocking %’, depending on the test properties and the settings of the technical equipment used for the readout. Routinely appropriate positive and negative control samples are included, and most-times samples are tested in multifold. Standardisation is obtained by including (a dilution range of) a defined reference sample, which also allows matching a certain score to pre-set values for determining positives or negatives, and allows correlation to a biological meaning, for example: an amount of antigen to potency, or an amount of antibody to a level of immune protection.

Many variants of an ELISA set-up are known, but typically these employ the immobilisation of an antigen or an antibody to a solid phase, e.g. to a well of a microtitration plate. When an antibody is immobilized the test is called a ‘capture’ or ‘sandwich’ ELISA. Next a test sample is added, allowing the ligand (e.g. an antigen or antibody to be detected) to bind. Then a detector (an antibody, antigen, or other binding component) is added which often is conjugated to a label, for instance to an enzyme that can induce a colour reaction, which can be read spectrophotometrically. Other types of label could be using luminescence, fluorescence, or radioactivity. The use of a labelled detector is intended to provide amplification of signal strength to enhance test sensitivity, however, it may also introduce background signal, reducing the signal to noise ratio.

In a variant of the ELISA protocol, the test specificity is improved by the introduction of a competitive binding, in which case the test is called a ‘competition-’, ‘inhibition-’, ‘interference-’, double-recognition, or ‘blocking ELISA’. In such an assay, a factor in the test sample (an antibody or an antigen) competes with a labelled detector antibody/antigen for binding to a molecule (antigen or antibody) immobilised to the solid phase. This causes a reduction in the maximal label signal, which is a sensitive way to measure presence or amount of the competing factor. The result can be expressed as a percentage of inhibition of the maximal ELISA signal.

General references to enzyme immunoassays exist in a variety of publications, among others in standard laboratory text books, such as: The Immunoassay Handbook (4th ed.: Theory and applications of ligand binding, ELISA and related techniques'; D. G. Wild edt., 2013, ISBN-10: 0080970370); and: The ELISA Guidebook' (Methods in Molecular Biology, vol. 149, J. R. Crowther, Humana Press, 2000, ISBN-10: 0896037282). Alternatives are manuals from commercial suppliers such as: “Technical guide for ELISA”, KPL Inc., Gaithersburg, Md., USA, 2013; and: “Assay guidance manual” by Eli Lilly &Co., chapter: Immunoassay methods, K. Cox et al., May 2012.

A general overview on detection and control of BVDV is: OIE, Manual of Diagnostic Tests and Vaccines for Terrestrial Animals 2015 (NN, Chapter 2.4.8).

In an embodiment the method for differentiating animals according to the invention, applies an ELISA. This can be of any type such as a blocking- or sandwich-ELISA. Such tests can be optimised and fine-tuned to the particular type of differentiation required. Also this may depend on the type of Pestivirus or type of antibody to be detected, and the type of animal test sample to be screened. The skilled person is perfectly capable of applying these techniques and making optimisations, to arrive at test results that are sufficiently specific and selective, to make the required differentiation of animals.

These methods now enable the effective application of the DIVA principle, and the organisation and performance of large-scale screening- and eradication programs.

Therefore in a further aspect the invention relates to a method for diagnosing an animal that had been vaccinated with a vaccine according to the invention, for an infection with a Pestivirus other than a mutant Pestivirus comprised in the vaccine, the method comprising the steps of:
obtaining a sample from said vaccinated animal, and
testing said sample for the presence of an antibody against a Pestivirus other than a mutant Pestivirus comprised in the vaccine, by using a mutant Pestivirus comprised in the vaccine, or a chimeric Erns protein as expressed by the mutant Pestivirus comprised in the vaccine, in an appropriate immuno-assay.

Evidently, the reverse also applies:

Therefore in an embodiment the method for diagnosing an infection according to the invention comprises the step of testing the sample for the presence of a Pestivirus other than a mutant Pestivirus comprised in the vaccine, with an antibody against an Erns protein, which antibody does not bind specifically with the chimeric Erns protein as expressed by the mutant Pestivirus that was comprised in said vaccine.

Methods for collection and preparation of samples are well known in the art. Such samples can be any type of biological sample in which sufficient amounts of the virus or of the antibody to be detected is present. Typically these samples can be: blood, serum, milk, semen, urine, faeces, or a tissue sample such as an ear-puncture.

What constitutes an “appropriate immuno-assay” e.g. for the detection of non-vaccine Pestivirus may depend on the particulars of the sample, the virus, or other parameters of the test to be performed. Selecting and optimising such a test is well within the routine capabilities of the skilled person. Typically the mutant Pestivirus according to the invention, or a chimeric Erns protein as expressed by the mutant Pestivirus can be used as detection antigen. The virus would be inactivated, and virus or chimeric Erns can for example be coated to a support matrix for use in such an immuno-assay.

In a preferred embodiment such an immuno-assay is an ELISA.

To facilitate the methods for differentiating and the methods for diagnosing, both according to the invention, the invention also provides the assembly and the use of a diagnostic test kit for implementing these methods.

Therefore in a further aspect the invention relates to a diagnostic test kit comprising a mutant Pestivirus according to the invention, or a chimeric Erns protein as expressed by the mutant Pestivirus.

In a preferred embodiment of a diagnostic test kit according to the invention, the mutant Pestivirus is comprised in inactivated form.

A “diagnostic test kit” relates to a kit of parts for performing the methods for differentiating, or the method for diagnosing, both according to the invention. The kit comprises one or more components for applying the methods, in particular: a mutant Pestivirus according to the invention, or a chimeric Erns protein as described for the invention. The mutant Pestivirus or chimeric Erns protein should be in a convenient form and container, optionally with buffers for sample dilution and incubation, blocking, or washing, and optionally instructions how to perform the method, and how to read- and interpret the results.

In an embodiment the kit may comprise a container having multiple wells, such as a microtitration plate. The wells of the container may be treated to contain one or more components for use in the methods according to the invention.

In a preferred embodiment of the diagnostic test kit according to the invention, a mutant Pestivirus according to the invention, or a chimeric Erns protein, is immobilized to the wells of a microtitration plate.

The instructions optionally comprised with the diagnostic test kit according to the invention, may for example be written on a box containing the constituents of the kit; may be present on a leaflet in that box; or may be viewable on, or downloadable from, an internet website from the distributor of the kit, etc.

For the invention, the diagnostic test kit may also be an offer of the mentioned parts (relating to commercial sale), for example on an internet website, for combined use in an assay comprising the methods according to the invention.

A diagnostic kit such as according to the invention, is also called a ‘companion diagnostic’, as it is specifically suitable for a use in combination with a marker vaccine, such as the vaccine according to the invention. With their combined use it is now possible to apply effective control programs for reducing the prevalence of wild type Pestiviruses in a population of animals.

Therefore, in a further aspect the invention relates to a method for controlling an infection with a wild type Pestivirus in a population of animals from the order of the Artiodactyla, by the combined use of a vaccine and a diagnostic test kit, both according to the invention.

The invention will now be further described by the following, non-limiting, examples.

### EXAMPLES

### Example 1

### Construction of Chimeric Erns Gene

A chimeric Erns gene was constructed for insertion into an existing cDNA clone of a BVDV vaccine virus. The chimeric Erns gene was assembled from the Erns gene from Bungowannah virus, but the 3′ part of the chimeric Erns gene was based on a BVDV-1 strain CP7 virus.

Methods and materials applied were essentially based on previous work described by Zemke et al. (2010, Vet. Microbiol., vol. 142, p. 69-80) and Richter et al. (2011, Virology, vol. 418, p. 113-122). Richter et al. describe a cDNA clone of Bungowannah virus.

Zemke et al. describe the construction of a cDNA clone of a BVDV-1 CP7 virus with Npro deletion. Virus rescued from this cDNA was used for vaccination of cattle, and was shown to be safely attenuated, and capable of providing an effective immune protection against a heterologous challenge with BVDV-2 virus.

In the cloning plasmids used herein, the cDNA's were flanked by a promoter, a start- and a stop-codon, and/or by useful restriction enzyme sites, when appropriate.

On the basis of the completely synthetic infectious cDNA clone pBVDV-Ib_synth_ΔNpro, the BVDV-1 CP7 Erns protein was substituted with the Bungowannah virus Erns protein. For a correct processing of the Bungowannah virus Erns protein and the in the polyprotein upstream localized E1 protein, the C-terminus of CP7 Erns, harboring a membrane anchor region and a transporter peptide, was retained

The Bungowannah virus Erns encoding region was amplified using primers Bungo_Erns_Ph_F (5′-CTTTCAAGTCACAATGGGAACCAACGTGACACAATGGAAC -3′) (SEQ ID NO: 7) and Bungo_Erns_oTP_R (5′-CGCGGTCCCTTGCCTGGCACTCTCTACTACCTCGGTGTAACCGTCAAC -3′) (SEQ ID NO: 8) as template DNA a synthetic plasmid Bungo_C-E2mod_pMK_RQ (Geneart).

The Bungowannah Erns gene was isolated from the Bungowannah cDNA construct (Richter, supra), by PCR using two primers: the plus-sense primer for the 5′ side of the Bungowannah Erns gene, starting from the end of the Capsid gene: Bungo_Erns_Ph_F: 5′-CTTTCAAGTCACAATGGGAACCAACGTGACACAATGGAAC-3′ (SEQ ID NO: 7), and the minus-sense primer for the region Erns CP7/Erns Bungowannah virus: BungoErns_oTP_R: 5′-CGCGGTCCCTTGCCTGGCACTCTCTACTACCTCGGTGTAACCGTCAAC-3′, (SEQ ID NO: 8). The 619 bp PCR fragment was inserted into plasmid pBVDV-Ib_synth_ΔN^{pro} by restrictions-free targeted cloning using Phusion Polymerase (New England Biolabs). The resulting cDNA construct was called: pBVDV-1CP7_ΔNpro_Erns-Bungo/CP7 (SEQ ID NO: 9).

Alternatively, a Bungowannah Erns gene could also have been obtained starting from a DNA copy of SEQ ID NO: 3, or a Bungowannah viral-genome isolate, using appropriate PCR or rtPCR primers.

All methods and materials applied were standard techniques, and used commercial kits and -tools according to the manufacturer's instructions, in short: cloning plasmids were amplified in Escherichia coli DH10B™ cells (Invitrogen). Plasmid DNA was purified by using Qiagen Plasmid Mini™ or Midi Kit. Sequencing was carried out using a Big Dye™ Terminator v1.1 Cycle sequencing Kit (Applied Biosystems). Nucleotide sequences were read with an automatic sequencer (3130 Genetic Analyzer™, Applied Biosystems) and analysed using the Genetics Computer Group software version 11.1 (Accelrys Inc.) and Genious™ software (Biomatters Ltd).

### Example 2

### Recovery and Amplification of Mutant Pestivirus

The newly formed cDNA construct pBVDV-1CP7_ΔNpro_Erns-Bungo/CP7 was used for in vitro RNA transcription of the SmaI linearised cDNA construct, performed by T7 RiboMax™ Large-Scale RNA Production System (Promega) according to the manufacturer's instructions. The amount of RNA was estimated by ethidium bromide staining after agarose gel electrophoresis. For transfections, 1×10̂7 KOP-R or MDBK cells or another suitable ruminant cell line, were detached using a trypsine solution, washed with PBS, mixed with 1-5 μg of in vitro synthesized RNA, and electroporated (two pulses at 850 V, 25 μF, 156 ω) using an Gene Pulser™ Xcell Electroporation System (Bio-Rad). For virus recovery, supernatants of the transfected cells were harvested at 72 h p.t. and inoculated into suitable ruminant cell lines. Infectious titers were determined for virus stocks as well as for growth-kinetics experiments. The identity of the recombinant viruses was confirmed by sequencing.

After incubation of the transfected cells, mutant BVDV Pestivirus could be obtained. Several clones were picked, and these were amplified in KOP-R or MDBK cells, for a number of passages, to select replicative clones, and amplify their titer.

Recombinant viruses from more than 20 clones were passaged, and after several passages virus titre was checked, either in cell-culture supernatant which was cleared by centrifugation or in cleared freeze-thawed sample of whole culture. Titers in supernatant were usually a little higher than in the freeze-thaw sample. Two recombinant viruses were selected, nrs. 1 and 10 for further study; at passage 20 these both grew to a titer of 1×10̂5 PFU/ml.

### Example 3

### Sequencing of Amplified Mutant Pestivirus

The two selected recombinant viruses BVDV-Ib_synth_dNpro_Erns_Bungowannah, clones nr. 1 and 10 were subjected to nucleotide sequencing of their full genome, to detect if any relevant mutations had occurred. Using a ‘next generation sequencing’ approach, sequencing was done at passage levels 13 and 19+1, and only a very limited number of mutations could be observed in comparison to the parental virus BVDV-Ib_synth_dNpro: clone 1 had no mutation causing an amino acid exchange, only one silent mutation at P13 in E1 at C 1531A; P19+1 had one silent mutation in the Bungowannah Erns gene C1156T and one in the NS5B gene at nt G10723A. Clone 10: had some point mutations causing an amino acid exchange: P13 contained a mixed population in capsid gene of G/C741 and E2 gene C/A 2431, whereas P19 had two point mutations, both causing an amino acid exchange in the NS2 gene at G3417C and G3968C, and 4 silent mutations, one in the NS3 gene G6748A, two in the NS4B gene at A7240C and A7348G, and one in the NSSA gene at A8908G.

Considering that these are RNA viruses, and seen the relatively high number of passages, these were very good results. Therefore, the genetic stability of the investigated recombinant viruses was sufficiently demonstrated and both were used for further investigations.

### Example 4

### Multistep Virus Growth Kinetics

To further characterize the BVDV-Ib_synth_ΔN^{pro}_E^{rns}_Bungowannah clones 1 and 10, multistep growth kinetics were investigated.

KOP-R cells were inoculated with BVDV-Ib (Cp7), BVDV-Ib_synth_ΔN^{pro} (Cp7_ΔNpro), BVDV-Ib_synth_ΔN^{pro}_E^{rns}_Bungowannah (Cp7_ΔNpro_Erns Bungo) clones 1 and 10, at P23, with an m.o.i. of 0.1 for 2 h. The applied virus inocula were back titrated to determine the titers actually used in the experiments. After incubation, cells were washed twice, fresh medium was added and cells were frozen at 0, 24, 48, 72 and 96 hours post inoculation. After thawing, cleared cell culture supernatants were titrated on MDBK-cells to determine the virus titers for each time point. Virus was detected by immunofluorescence staining with an antibody specific for BVDV NS3 protein: monoclonal antibody WB103/105 (available from APHA Scientific, New Haw, Addlestone, Surrey, UK), and the titers were calculated and expressed in TCID50/ml. The experiment was performed twice and results of one representative experiment are presented in FIG. 1.

Erns BVDV_synCp7_ΔNpro_Erns Bungo clones 1 and 10 viruses demonstrated a reduced growth when compared to wild-type virus BVDV-1 CP7, but their replication was only slightly impaired in comparison to the recombinant BVDV vaccine virus BVDV1 CP7_ΔNpro.

### Example 5

### Serological Testing of Mutant Pestivirus Vaccine in Cattle—Inactivated Vaccine

Clone 10 virus was amplified further until passage 21. Culture supernatant was harvested and stored until use. The titre of clone 10 virus (construct: BVDV-Ib_synth_ΔNpro_Erns_Bungowannah) had by then improved to 4.6×10̂5 PFU/ml. The viral genomic identity was verified again by genome sequencing. This virus material was used for testing its capacity to induce a seroresponse in cattle.

The following virus-constructs were compared:
BVDV-Ib_synth_ΔNpro (dNpro): a BVDV vaccine strain, with attenuating deletion of Npro (except for the 12 N-terminal amino acids)
BVDV-Ib_Erns_Bungowannah (Erns-Bungo): a mutant Pestivirus according to the invention, comprising a chimeric gene according to SEQ ID NO: 5
clone 10 virus (in short: dNpro_Erns Bungo), also a mutant Pestivirus according to the invention, comprising next to a chimeric gene according to SEQ ID NO: 5, a deletion of Npro (except for N-term. 12 aa)
wild type Bungowannah virus (BungoV).

### 5.1 Virus Antigen Preparation

Virus-culture supernatants from all four viruses was obtained and inactivated using BEI. The inactivation was verified in two passages on cells, and tested by immuno-fluorescence using antisera against BVDV NS3 and Bungowannah Erns. No virus growth could be detected, confirming complete inactivation.

### 5.2 Vaccinations:

For each virus, 4 ml BEI-inactivated viral antigen was mixed with 1 ml Polygen™ adjuvant, to a final concentration of 12% v/v. This mixture was administered by intramuscular injection at 1 ml/dose to a calf; one calf per antigen. The inactivated vaccine antigen content was the pendant of between 10̂7 and 10̂8 TCID50/ml, except for clone 10 virus antigen, which had lower antigen content at between 10̂5-10̂6 TCID50/ml, for the three vaccinations.

The vaccination schedule was: day 0: 1^{st} vaccination; day 21: 1^{st} booster vaccination; day 42: 2^{nd} booster vaccination. Blood samples were collected at days: 0, 21, 28, 35, 42, and 49. Serum from these samples was then tested in different assays.

### 5.3 Serological Testing

### 5.3.1 Virus Neutralisation Assay

A serum neutralisation assay was performed to determine the strength of the neutralizing antibodies induced against BVDV-Ib and/or Bungowannah virus.

The cattle sera were diluted in Log 2 steps in a 96 well micro-titration plate, and incubated with 30-300 TCID50/ml of the live virus BVDV or Bungowannah, for 1 h at 37° C. Subsequently, 1×10̂5 MDBK cells per well were added. The neutralizing dose 50% (ND50) per ml was determined 3 days post incubation, by immunofluorescence staining of the cells using a monoclonal antibody against Pestivirus NS3 protein, recognising both BVDV and Bungowannah viruses: WB112 (APHA Scientific).

Results are presented in FIG. 2, and demonstrate that both mutant Pestiviruses could induce good BVDV neutralising titers, approaching those induced by the BVDV vaccine strain (dNpro). Although the titers from the clone 10 vaccine were initially low, because of the lower antigen content. This improved quickly after the first booster. Bungowannah virus could not induce anti-BVDV antibodies.

With respect to anti-Bungowannah antibodies, these were only induced by the Bungowannah virus vaccine; indicating that even the expression of a large part of the Bungowannah Erns gene in the mutant Pestiviruses Erns-Bungo and clone 10, did not induce Bungowannah neutralising antibodies.

### 5.3.2 Immunofluorescence Inhibition Assay

An Immunofluorescence inhibition (IFI) assay was developed to allow flow cytometry experiments.

The IFI was performed essentially as described in Beer et al. (2000, Vet. Microbiol., vol. 77, p. 195-208). In short: MDBK-cells were infected with BVDV strain NCP7. Three days after inoculation, the cells were harvested, fixed with 4% paraformaldehyde for 15 min. at room temperature. Subsequently, the cells were permeabilised for 5 min. with 0.01% digitonin at room temperature, and washed three times with FACS buffer. 1×10̂5 of these cells were incubated with 100 μl of the cattle sera from the inactivated vaccinations, diluted 1:2 in FACS buffer, or in only FACS buffer, for 1 h. Next, a monoclonal antibody specific for BVDV Erns protein: WB210 (APHA Scientific) was diluted 1:100, and was added and incubated for 10 min. Thereafter, the cells were washed three times and 100 μl of a commercial goat anti-mouse antibody conjugated with ALEXA488 marker was added and incubated for 5 min. After three washing steps, flow cytometry analysis was performed using a FACSscan™ Cytofluorometer and the software CellQuest (both: Becton Dickinson).

The number of infected cells was determined by anti-NS3 staining (WB112) and was found to be 100%. The IFI-values were determined by measuring the median fluorescence intensity (MFI) for WB210-specific binding. The median fluorescence intensity for the staining of uninfected control cells by Bungo_Erns was set to 100% inhibition (no Bungo_Erns present, detected fluorescence intensity was set as background); the MFI for Bungo_Erns-specific antibody staining of BungoV-infected cells was set to 0% inhibition (Bungo_Erns was fully accessible for the detection antibody; normalized to the control). The % inhibition was determined as=(MFI NCP7 WB210-MFI sample)/MFI NCP7*100 and normalized to control WB210.

Control samples tested were: a cattle serum immunized with BVDV-Id (PC), and a serum negative for BVDV-specific antibodies (NC).

Results of the IFI assay are presented in FIG. 3, and showed that only the positive control sera: WB210 and PC, as well as the serum against the BVDV vaccine (dNpro) from day 49, displayed an IFI signal above the cut off value (80% inhibition of BVDV-Erns WB210). Other sera tested were negative, or below cut off.

This proves that no anti-BVDV-Erns antibodies were induced by any of the vaccines of the mutant Pestiviruses (Erns-Bungo or clone 10), only by a BVDV vaccine itself (dNpro).

### 5.3.3 Competition ELISA

To be able to determine the level of antibodies specific for BVDV Erns that were induced, a competition ELISA for BVDV Erns was established, on the basis of the commercially available anti-BVDV Erns monoclonal antibody WB210. This was compared with a commercial total anti-BVDV antibody test.

Reagents and plates were taken from the commercially available BVDV total Ab™ kit (Idexx). Antibodies used in this assay were WB210 and a goat-anti-mouse-POD (Dianova). 100 μl sample diluent was applied to each well, and the commercial antibodies were diluted in PBS-WB210 1:400. These antibodies and cattle sera from the inactivated-vaccine trial were mixed in a separate tube, added to the plate and incubated for 90 min. at room temperature. The plates were washed 5 times with PBS+0.1% Tween™20. The secondary antibody-conjugate goat-anti-mouse-POD was diluted in TBS+2% skimmed milk powder+2% fish gelatin 1:1000. 100 μl was added per well and incubated for 60 min. at 37° C. After that second incubation, the plate was washed again 5 times with PBS and dried properly. 100 μl of TMB substrate was applied per well and incubated. Stop solution was added after 3 min. The absorbance was measured at 450 nm. The % inhibition value was calculated as follows: (OD Ab pure−OD sample)/OD Ab pure*100.

The commercially available BVDV total Ab™ indirect ELISA (Idexx) was conducted according to the manufacturer's instructions.

Cattle sera from the initial bleeding and 49 days post first immunization (dpi) were tested for the presence of BVDV Erns- and for total BVDV-specific antibodies.

Control sera were: cattle sera from previous animal trials: anti-BVDV (PC), anti-Bungowannah (PC Bungo), and anti-BVDV negative (NC).

Results are presented in FIG. 4 and indicate that—as expected—only serum of the animal immunized with the BVDV vaccine (dNpro) inhibited the binding of the BVDV Erns-specific antibody WB210.

Whereas, general BVDV-specific antibodies were found in sera of animals immunized with the BVDV vaccine, and with both mutant Pestiviruses Erns-Bungo or clone 10, but not with Bungowannah virus.

### 5.4 Conclusions

The immunisation with BEI-inactivated virus antigens of mutant Pestiviruses according to the invention: clone 10 and Erns-Bungo, both induced robust levels of anti-BVDV-specific neutralising antibodies. Because it is known that BVDV-neutralising antibodies, and at these levels, are correlated to protection against BVDV infection and -disease, therefore these results demonstrated the capacity of these mutant Pestiviruses to perform as effective inactivated vaccines against BVDV infection and disease.

In addition, no BVDV Erns-specific antibodies were found in the different assays performed, demonstrating that these mutant Pestiviruses enable a very distinct marker-screening based on BVDV Erns protein, upon their use in BVDV vaccines.

### Example 6

### Serological Testing of Mutant Pestivirus Vaccine in Cattle—Live Vaccine

A further study of the vaccine- and marker-capacity of the mutant Pestiviruses according to the invention: clones 1 and 10 was conducted in cattle, this time as live virus vaccines.

### 6.1 Preparation of Virus Antigens

BVDV-Ib_synth_ΔNpro_Erns_Bungowannah clones 1 and 10 were grown on KOP-R cells in the presence of IFN inhibitor A. Cleared culture supernatant was harvested at 3 days post inoculation, and titrated on MDBK cells. The titers were determined by immunofluorescence staining with moab WB103/105 for NS3. The titers for passage 19+1 were quite comparable for clone 1 and 10 viruses: respectively 2.2 and 4.6×10̂6 TCID50/ml. Their genetic stability had been demonstrated previously.

### 6.2 Vaccinations

Four calves of about 6 months of age were immunized: 2 with clone 1 and two with clone 10 virus. Inoculum doses at the three vaccination dates, were: about 1×10̂7 for clone 10 virus, and about 3×10̂6 for clone 1 virus.

The vaccination schedule was: day 0: 1^{st} vaccination; day 21: 1^{st} booster vaccination; day 42: 2^{nd} booster vaccination. Blood samples were collected at days: 0, 21, 28, 43, and 49. Serum from these samples was then tested in different assays.

### 6.3 Serological Testing

### 6.3.1 Neutralising Antibodies

The production of neutralising antibodies specific for BVDV or Bungowannah virus was determined in virus-specific serum neutralisation assays as described above; results are presented in FIG. 5.

The first immunisation induced low neutralizing antibody titers (1.5-16 ND50/ml) against BVDV, whereas a strong booster effect was seen after the 1st booster vaccination, increasing the anti-BVDV titers up to 256 to 1024 ND50/ml. The 2nd booster vaccination did not further increase the titers markedly.

No significant neutralizing antibodies specific for Bungowannah virus were detected over time.

### 6.3.2 BVDV NS3 Specific Antibodies

Commonly in cattle vaccinated against BVDV, the neutralizing antibodies are mainly directed against the viral protein E2. Non-neutralising antibodies are also produced against NS3 (p80) and Erns. However, significant levels of anti-NS3 antibodies are only induced upon the presence of a replicating virus, not by inactivated virus antigens. To determine the level of NS3-specific antibodies induced in these trials using live vaccines. The BVDV p80 antibody™ competition ELISA kit (IDvet, France) was used, following the manufacterer's instructions.

Results are presented in FIG. 6, indicating that at 3 weeks after the 1^{st} booster vaccination (day 42 post first immunization) all four animals were found to be clearly positive for N53-specific antibodies.

### 6.3.3 Competition ELISA

In order to determine the level of antibodies specific for BVDV Erns, as proof of suitability for a DIVA approach, a competition ELISA for BVDV Erns was conducted, using WB210 moab. Test set-up and performance was as described above.

Results are presented in FIG. 7. Most importantly: no BVDV Erns-specific antibodies were found in the sera of cattle immunized with live BVDV-Ib_synth_ΔNpro_Erns_Bungowannah clones 1 or 10.

Nevertheless, the presence of total BVDV-specific antibodies was confirmed using the BVDV total Ab™ indirect ELISA (Idexx). As an indication, the S/P value of animal 2 clone 10 was 0.27, only just below cut-off.

### 6.4 Conclusions

Like for the results of the vaccination with inactivated vaccines made of mutant Pestiviruses according to the invention, also the vaccination with live vaccines from these mutant Pestiviruses demonstrated effective vaccination and excellent marker functionality.

This because a robust BVDV-specific antibody response was induced when cattle were immunized with live BVDV-Ib_synth_ΔNpro_Erns_Bungowannah viruses clone 1 or clone 10. This was demonstrated using antibodies that were: NS3-specific, BVDV-neutralizing, and total BVDV-specific. This confirmed the suitability as vaccine against BVDV, both as live- or as inactivated virus.

Importantly, no antibodies specific for BVDV Erns could be detected in these sera, using a competition ELISA with a BVDV Erns-antibody, confirming clearly the feasibility of the DIVA marker principle.

### Example 7

### Further Experiments in Progress

Further tests in experimental animals are in planning. Currently in progress is a trial in groups of young calves, 6 months of age, each group with 5 animals. The experiment will test different vaccination regimens for a live attenuated mutant Pestivirus according to the invention: by single- or dual shot vaccination. For comparison a live attenuated vaccine strain of BVDV will be tested alongside, to compare the attenuating effect of the attenuating Npro deletion itself, in a single shot schedule. The Npro deletion mutant does still possess the N-terminal 12 amino acids of Npro, just as that is the case for the mutant Pestivirus tested here.

Combined the different groups will be assigned as follows:
1. non vaccinated controls
2. BVDV-Ib_synth_ΔNpro, single shot
3. BVDV-Ib_synth_ΔNpro_Erns_Bungo, clone 1, single shot
4. BVDV-Ib_synth_ΔNpro_Erns_Bungo, clone 1, priming and booster vaccination.

The experiments' time schedule is: day 1: first vaccination; day 21: second vaccination (group 4); day 42: BVDV-1b challenge; and day 70: end of trial.

There will be serum sampling at weekly intervals. Vaccination dose will be 1×10̂6 TCID50, by intra-muscular route. Challenge will be by intranasal route, using a dose of 2×10̂6 TCID50 BVDV-Ib SE5508.

Take of the vaccine and of the challenge will be checked by viraemia: after the 1st vaccination and after the challenge, by monitoring of purified leukocytes, and testing for nasal shedding of vaccine- or challenge virus (differentiation by serology and/or PCR) for up to 14 days consecutive days.

### LEGEND TO THE FIGURES

FIG. 1: Multistep growth curve kinetics of BVDV-Ib_synth_ΔN^{pro}_E^{rns}_Bungowannah (Cp7_ΔNpro_Erns Bungo) clones 1 and 10, at P23, in comparison to recombinant parent viruses BVDV-Ib (Cp7) and BVDV-Ib_synth_ΔN^{pro} (Cp7_ΔNpro). KOP-R cells were infected with an m.o.i. of 0.1.

FIG. 2: Results of serum-neutralisation assays following inactivated vaccinations. Numbers (352, 353, 365, and 321) are calf ear-tag numbers. Broad arrows indicate days of vaccination/booster.

FIG. 3: Immunofluorescence inhibition (IFI) assay. Median fluorescence intensity values were normalized to the control staining of uninfected cells (control WB210). Mean values and standard derivations of two experiments are shown. PC is a cattle serum immunized with BVDV-Id, and NC is a serum negative for BVDV-specific antibodies.

FIG. 4: Results of ELISA assays for BVDV-specific antibodies in cattle sera from inactivated-antigen vaccination trial.

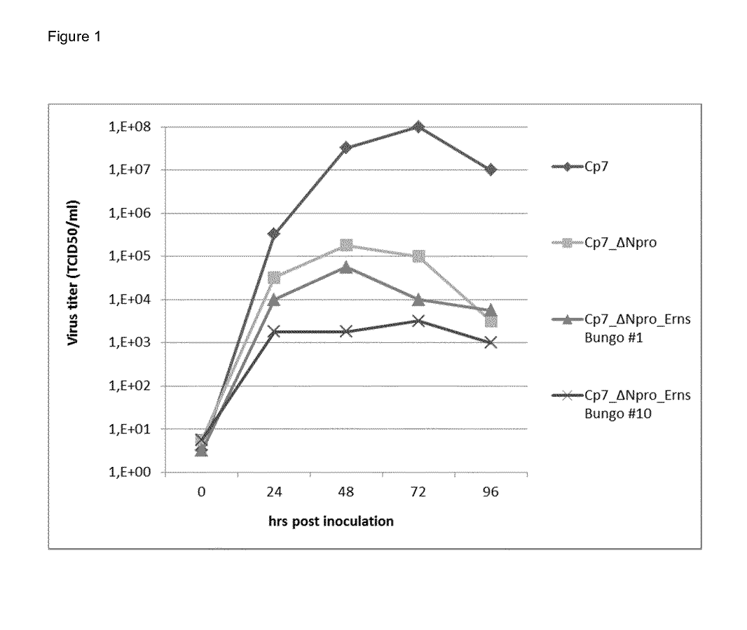

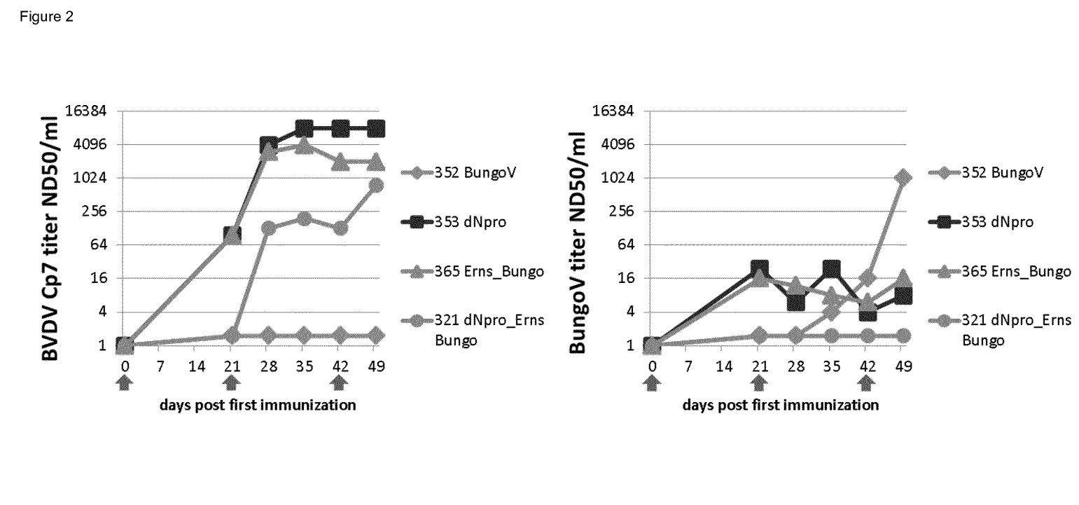

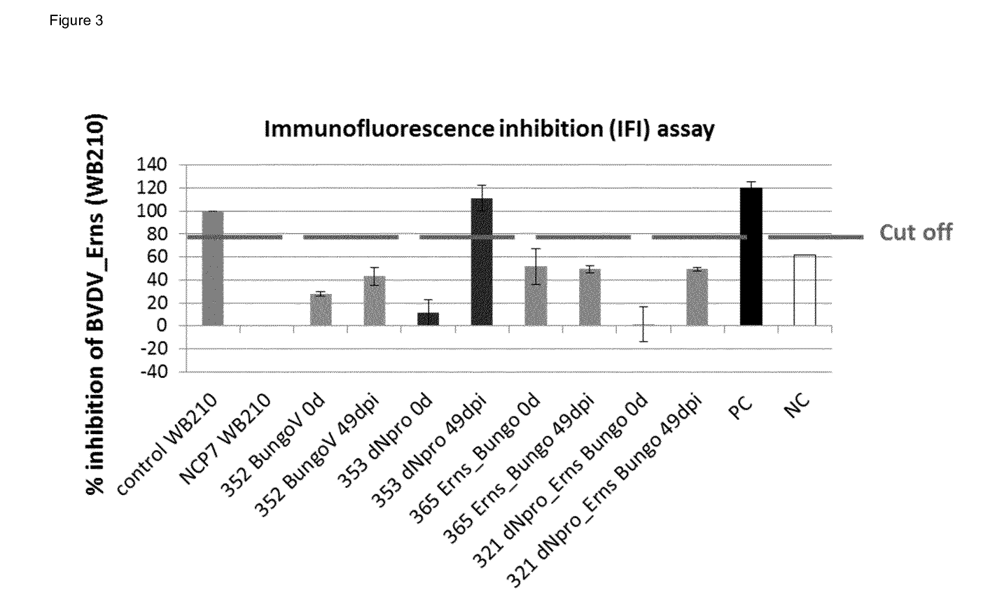

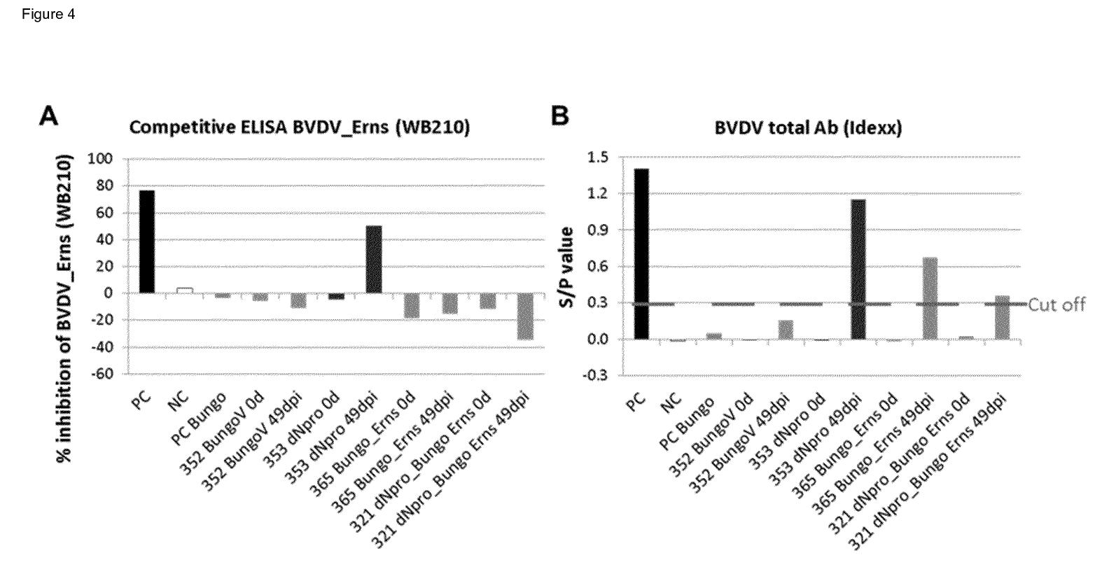

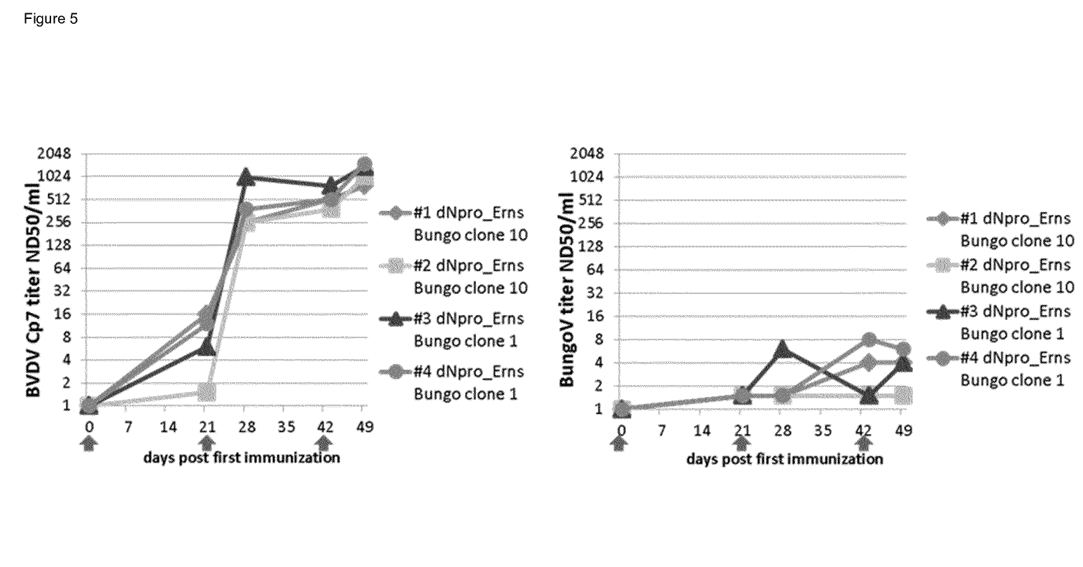

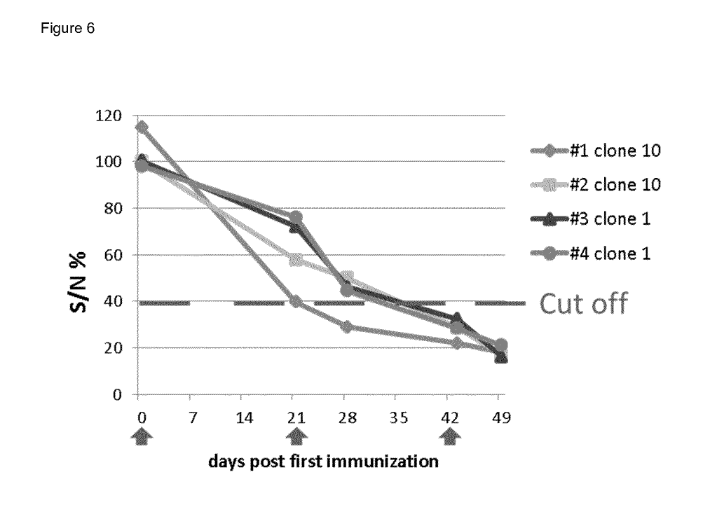

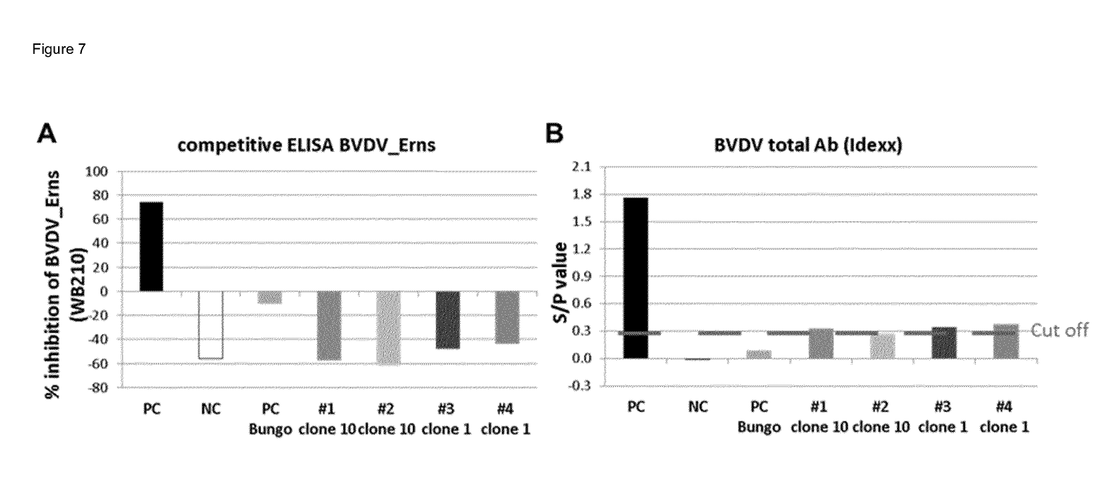

Panel A: competition Elisa for BVDV Erns antibodies: in the presence of BVDV_Erns specific antibodies in cattle sera, the binding of WB210 (BVDV Erns specific antibody) to immobilized BVDV is inhibited and detected in the competition ELISA. Sera from cattle immunized with constructs carrying a Bungo-Erns were not able to block binding of BVDV Erns-antibody WB210.

Panel B: total anti-BVDV antibodies: BVDV total Ab indirect ELISA (Idexx) was used to detect total BVDV-specific antibodies in the cattle sera. Samples were considered positive if S/P values were greater than 0.3.

0d=initial bleeding before first immunization; 49dpi=49 days post first immunization.

FIG. 5: Results of serum neutralization assays, to determine neutralizing antibodies against BVDV and against Bungowannah virus in sera of cattle vaccinated with live mutant Pestiviruses according to the invention: clone 1 clone 10. The broad arrows indicate the days of immunization.

FIG. 6: Detection of NS3-specific antibodies induced by live virus vaccination with live mutant Pestiviruses according to the invention: clone 1 and clone 10. Detection was by using the BVDV p80 antibody™ competition ELISA kit (IDvet) over time. The broad arrows indicate the days of immunization. Samples were considered positive if S/N % values were lower than 40%.

FIG. 7: Results of the detection of BVDV-specific antibodies in cattle sera at 49 days post vaccination with live mutant Pestiviruses according to the invention: clone 1 and clone 10.

Panel A: In the presence of BVDV Erns specific antibodies in cattle sera, the binding of moab WB210 (BVDV Erns specific) to immobilized BVDV is inhibited, as detected in a competition ELISA. Both clones 1 and 10 did not induce detectable BVDV Erns-specific antibodies, using moab WB210 as detector.

Panel B: Results of the BVDV total Ab™ ELISA, detecting total anti-BVDV-specific antibodies in the cattle sera. Samples were considered positive if S/P values were greater than 0.3. Control cattle sera: BVDV positive (PC), Bungowannah positive (PC Bungo), and BVDV negative (NC).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. A mutant Pestivirus comprising a genome wherein an Erns gene is mutated, wherein said mutated Erns gene is a chimeric Erns gene, wherein said chimeric Erns gene comprises a 5′ part and a 3′ part, wherein said 5′ part comprises 60-95% of the chimeric Erns gene, wherein said 3′ part comprises the remainder of the chimeric Erns gene, wherein said 5′ part comprises the corresponding part of an Erns gene from a Pestivirus that is genetically distant from the mutant Pestivirus, and wherein said 3′ part comprises the corresponding part of an Erns gene from a Pestivirus that is genetically close to the mutant Pestivirus.

23. The mutant Pestivirus of claim 22, wherein the mutant Pestivirus is a Pestivirus selected from the group consisting of: bovine viral diarrhoea virus (BVDV), classical swine fever virus (CSFV), and border disease virus.

24. The mutant Pestivirus of claim 22, wherein the Erns gene from a Pestivirus that is genetically distant from the mutant Pestivirus, is an Erns gene from a Pestivirus selected from the group consisting of: Antelope Pestivirus, Bungowannah virus, Norway rat Pestivirus, atypical porcine Pestivirus (APPV), and Rhinolophus affinis Pestivirus.

25. The mutant Pestivirus of claim 22, wherein the Erns gene from a Pestivirus that is genetically close from the mutant Pestivirus, is an Erns gene from a Pestivirus selected from the group consisting of: bovine viral diarrhoea virus type 1 (BVDV-1), bovine viral diarrhoea virus type 2 (BVDV-2), CSFV, border disease virus, Reindeer Pestivirus, Giraffe Pestivirus, and HoBi Pestivirus.

26. The mutant Pestivirus of claim 22, wherein the mutant Pestivirus is an attenuated Pestivirus.

27. The mutant Pestivirus of claim 26, wherein the attenuated Pestivirus comprises a genome wherein the Npro gene is mutated.

28. The mutant Pestivirus of claim 22, wherein the mutant Pestivirus is a bovine viral diarrhoea virus (BVDV), wherein the BVDV is of the cytopathogenic biotype.

29. A method for the construction of the mutant Pestivirus of claim 22, wherein said method comprises mutating the Erns gene in a Pestivirus genome into a chimeric Erns gene as defined in claim 22.

30. A host cell comprising the mutant Pestivirus of claim 22.

31. A host cell constructed by the method of claim 29.

32. A vaccine for animals comprising the mutant Pestivirus according to claim 22 and a pharmaceutically acceptable carrier.

33. A vaccine for animals comprising the host cell of claim 30 and a pharmaceutically acceptable carrier.

34. A vaccine for animals comprising the host cell of claim 31 and a pharmaceutically acceptable carrier.

35. A method for the preparation of a vaccine for animals comprising the mutant Pestivirus according to claim 22 and a pharmaceutically acceptable carrier, wherein said method comprises:
a. infecting a culture of host cells with said mutant Pestivirus of claim 22,
b. incubating said infected culture of host cells,
c. harvesting said culture of host cells or a part thereof, and
d. admixing the culture of host cells or the part thereof, with a pharmaceutically acceptable carrier.

36. The mutant Pestivirus of claim 22 for use in a vaccine for animals.

37. The host cell of claim 30 for use in a vaccine for animals.

38. The host cell of claim 31 for use in a vaccine for animals.

39. A method for the prevention or reduction of an infection by a Pestivirus or of associated signs of disease in animals, wherein said method comprises administering the vaccine of claim 32 to said animals.

40. A method of vaccinating animals for the prevention or reduction of an infection by a Pestivirus or of associated signs of disease, wherein said method comprises administering the vaccine of claim 32 to said animals.

41. A method for differentiating animals vaccinated with the vaccine according to claim 32, from animals infected with a Pestivirus other than a mutant Pestivirus comprised in the vaccine, the method comprising the use of an antibody against an Erns protein, which antibody does not bind specifically with the chimeric Erns protein expressed by a mutant Pestivirus that is comprised in the vaccine.

42. A method of claim 41, wherein said antibody binds specifically with an Erns protein from a Pestivirus selected from the group consisting of: BVDV-1, BVDV-2, CSFV, and border disease virus.

43. A method for diagnosing an animal that was vaccinated with the vaccine according to claim 32, for an infection with a Pestivirus other than a mutant Pestivirus comprised in the vaccine, the method comprising the steps of:
a. obtaining a sample from said vaccinated animal, and
b. testing said sample for the presence of an antibody against a Pestivirus other than a mutant Pestivirus comprised in the vaccine, by using a mutant Pestivirus comprised in the vaccine, or a chimeric Erns protein as expressed by the mutant Pestivirus comprised in the vaccine, in an immuno-assay.

44. A diagnostic test kit comprising the mutant Pestivirus according to claim 22, or a chimeric Erns protein as expressed by the mutant Pestivirus.

45. A method for controlling an infection with a Pestivirus in a population of animals from the order of the Artiodactyla, comprising the use of the vaccine according to claim 32.

46. A method for controlling an infection with a Pestivirus in a population of animals from the order of the Artiodactyla, comprising the use of the diagnostic test kit according to claim 44.
